SEQUENCE LISTING (1 sequence)

SEQ ID NO: 1 - protein, 798 aa, Human
MNLQPIFWIGLISSVCCVFAQTDENRCLKANAKSCGECIQAGPNCGWCTNSTFLQEGMPTSARCDDLEALKKKGCPPDDIENPRGSKDIKKNKNVTNRSKGTAEKLKPEDITQIQPQQLVLRLRSGEPQTFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSAVGTLSANSSNVIQLIIDAYNSLSSEVILENGKLSEGVTISYKSYCKNGVNGTGENGRKCSNISIGDEVQFEISITSNKCPKKDSDSFKIRPLGFTEEVEVILQYICECECQSEGIPESPKCHEGNGTFECGACRCNEGRVGRHCECSTDEVNSEDMDAYCRKENSSEICSNNGECVCGQCVCRKRDNTNEIYSGKFCECDNFNCDRSNGLICGGNGVCKCRVCECNPNYTGSACDCSLDTSTCEASNGQICNGRGICECGVCKCTDPKFQGQTCEMCQTCLGVCAEHKECVQCRAFNKGEKKDTCTQECSYFNITKVESRDKLPQPVQPDPVSHCKEKDVDDCWFYFTYSVNGNNEVMVHVVENPECPTGPDIIPIVAGVVAGIVLIGLALLLIWKLLMIIHDRREFAKFEKEKMNAKWDTGENPIYKSAVTTVVNPKYEGK

(19) United States
(12) Patent Application Publication
(10) Pub. No.: US 2017/0369578 A1
(43) Pub. Date: Dec. 28, 2017

(54) REGENERATION OF AGED SATELLITE CELLS

(71) Applicant: CARNEGIE INSTITUTION OF WASHINGTON, Washington, DC (US)

(72) Inventors: Chen-Ming FAN, Washington, DC (US); Michelle ROZO, Washington, DC (US); Liangji LI, Washington, DC (US)

(21) Appl. No.: 15/634,298

(22) Filed: Jun. 27, 2017

Related U.S. Application Data
(60) Provisional application No. 62/355,491, filed on Jun. 28, 2016.

(51) Int. Cl. C07K 16/28; A61K 38/39; A61K 39/00

(52) U.S. Cl. CPC C07K 16/2842; A61K 38/39; C07K 2317/75; A61K 2039/505

(57) ABSTRACT

Methods and compositions described herein are useful for rejuvenating skeletal muscle stem cells (i.e., satellite cells), promoting skeletal muscle regeneration, improving exercise endurance, regenerating skeletal muscle degeneration associated with an age-related disorder of skeletal muscle, and treating, preventing, or reversing skeletal muscle conditions.

The present application claims benefit of U.S. Provisional Application No. 62/355,491, filed Jun. 28, 2016, the entire contents of which is incorporated herein by reference.

### GOVERNMENT SUPPORT

This invention was made with government support under the federal grant number R01 AR060042 awarded by the National Institute of Arthritis and Musculoskeletal and Skin Diseases, of the National Institutes of Health. The Government has certain rights in the invention.

### BACKGROUND

Age-dependent dysfunction in adult stem cells is attributable to both cell-intrinsic and -extrinsic inputs. Critical mechanisms underlying the functional decline of aged stem cells remain elusive. Accordingly, there exists a need to identify factors that are able to promote or reverse age-associated changes in skeletal muscle.

### SUMMARY

The methods and compositions described herein are useful for rejuvenating skeletal muscle stem cells (i.e., satellite cells), promoting skeletal muscle regeneration, improving exercise endurance, regenerating skeletal muscle degeneration associated with an age-related disorder of skeletal muscle, and treating, preventing, or reversing skeletal muscle conditions.

Interactions between stem cells and their microenvironment, or niche, are essential for stem cell maintenance and function. Knowledge of the niche for skeletal muscle stem cell, i.e. the satellite cell (SC), is incomplete. The presently disclosed technology demonstrates that β1-integrin is an essential niche molecule that maintains SC homeostasis, and sustains the expansion and self-renewal of this stem cell pool during regeneration. It is also demonstrated herein that β1-integrin cooperates with fibroblast growth factor 2 (FGF-2), a potent growth factor for SCs, to synergistically activate their common downstream effectors Erk (extracellular signal-regulated kinases) and Akt (protein kinase B). Importantly, SCs in aged mice display altered β1-integrin activity and insensitivity to FGF-2. Augmenting β1-integrin activity with a monoclonal antibody, for example, is demonstrated herein to restore FGF-2 sensitivity and improve regeneration after experimentally-induced muscle injury.

The presently disclosed technology provides methods of rejuvenating skeletal muscle satellite cells, especially in their microenvironment, or niche, in a subject in need thereof, comprising administering to the subject a composition or treatment which increases the level of activated β1-integrin in the subject in the location of the microenvironment of the satellite cells. Compositions of the presently disclosed technology may be used for increasing the frequency or number of a subject's skeletal muscle stem cells or satellite cells, increasing the size of regenerating myofibers, and/or increasing the efficiency of myogenic colony formation, thereby rejuvenating the skeletal muscle stem cells or satellite cells in the subject.

The presently disclosed technology provides compositions for and methods of promoting skeletal muscle regeneration in a subject in need thereof, that include administering to the subject a composition which increases the level of activated β1-integrin in the subject, wherein administration of the composition increases the frequency or number of the subject's skeletal muscle stem cells or satellite cells, especially in the microenvironment of the niche, and/or increases the size of regenerating myofibers of the subject, and/or increases the efficiency of myogenic colony formation of the subject, thereby promoting skeletal muscle regeneration in the subject.

### DESCRIPTION OF THE FIGURES

The patent or application file contains at least one drawing executed in color. Copies of this patent or patent application publication with color drawing(s) will be provided by the Office upon request and payment of the necessary fee.

FIG. 1. Young Itgb1^{-/-} SCs are defective in maintaining quiescence and sustaining regeneration. (a) Control and Itgb1^{-/-} SCs on myofibers (assay scheme in FIG. 7a) stained for Pax7, β1-integrin, and laminin (dotted line); arrows, basal β1-integrin in SC; arrowheads, muscle β1-integrin; asterisks, myonucleus; Scale bar, 5 μm. (b-d) Long-term tracing of β-gal lineage-marked (R26R^{LacZ} reporter) control and mutant SCs after tmx regimen: (b) X-gal reacted (blue) and nuclear fast red stained muscle sections at 7 and 180 d (day); arrows, SCs; asterisks, X-gal⁺ myofibers. (c) Quantified blue fibers per field using data in (b). (d) SC to fiber ratios at 7, 21, 90, and 180 d; n=3 animals per group, ten sections scored per animal. (e) Percentages of BrdU⁺YFP⁺/total YFP⁺ cells of control and Itgb1^{-/-} muscles after 1 month of BrdU administration; n=3 animals per group, ten sections scored per animal. (f) Percentages of MyoD⁺YFP⁺/YFP⁺ cells from the same animals in e. (g) Representative images (n≧15 per condition) of X-gal reacted control and Itgb1^{-/-} muscle sections at 5 d post CTX injury (scheme in FIG. 9a); Scale bar, 25 μm. (h,i) Average fiber number per field (0.228 μm²) (h) and mean fiber diameter (i) at 5 d and 10 d post injury; n=3 animals per group, 20 sections scored per animal. All numerical data are presented as mean±s.d.; Student's t-test: *P<0.05, **P<0.01, and n.s., not significant.

FIG. 2. Young Itgb1^{-/-} SCs are defective in maintaining proliferation and prone to differentiation. (a) EdU incorporation of YFP control and mutant cells in muscle sections 3 d after injury; Scale bar, 50 Lm. (b) Quantification of EdU⁺YFP⁺/YFP⁺ on d 2-5 daily; data are expressed as mean±s.d., n=3 animals per d; ten sections per sample; Student's t-test: *P<0.05, **P<0.01, and n.s., non-significant. (c) Western blot of FACS isolated control and Itgb1^{-/-}YFP⁺ SCs cultured for 72 h, using antibodies to proteins indicated. (d-f) FACS-aided cell cycle analyses using DNA content (stained by PI) of control and mutant SCs at 24, 48, and 72 h: (d) PI profiles at 72 h, (e) pie charts summarize cell fractions in G1, S, and G2/M, and (f) percentage deviation plot of mutant vs. control cells in cell cycle phases at stipulated time points; ModFit LT V2.2.11 was used to analyze percentages of each phase of the cell cycle. All data were determined to have “good” RCS, measurement of fit. (g) Fold changes of muscle differentiation genes upregulated in Itgb1^{-/-} SCs compared to control SCs at 72 h. All listed genes display “yes” significance (q<0.05) by Cuffdiff2, Genes were included only if one (control or Itgb1^{-/-}) had FPKM≧5 to control for elevated fold changes of genes with minimal expression.

FIG. 3. Itgb1^{-/-} SCs show a compromised response to FGF-2 that can be partially restored by exogenous FGF-2. (a) Control and Itgb1^{-/-} sections 10 d after injury stained for Pax7 and YFP; arrows, Pax7⁺ SCs; Scale bar, 25 μm. (b) Average number of Pax7⁺YFP⁺ cells per field (0.228 μm²) during regeneration; n=3 animals per time point, ten sections per animal; data are expressed as mean±s.d.; Student's t-test: *P<0.05; **P<0.01. (c) Western blots for pErk1 and pErk2, Erk1 and Erk2, pAkt, and Akt of control and Itgb1^{-/-} cells. Fibronectin addition (+) and FGF-2 concentrations (FGF(ng/ml)) are indicated. (d) Fold induction from data in c, normalized to control cells without fibronectin and FGF-2 (set at 1 arbitrary unit (a.u.)); n=4 parallel sets of myoblasts. Paired comparisons with significant differences are indicated; data are expressed as mean±s.e.m.; two-way ANOVA: *P<0.05. (e) Control and Itgb1^{-/-} SCs cultured for 96 h with or without 10 ng/ml FGF-2 were stained for YFP, Pax7, MyoD, and DAPI; arrows, Pax7⁺ cells, asterisks, Pax7⁺MyoD⁺ cells, and triangles, MyoD⁺ cells; scale bar=25 μm. (f) Percentage distribution of various cell populations from data in (e); n=3 animals; ≧20 myofibers per condition; two-way ANOVA: P<0.01 for Pax7⁺MyoD⁺ and MyoD⁺, −FGF vs.+FGF (control and Itgb1^{-/-}), control vs. Itgb1^{-/-} (−FGF and +FGF); P<0.05 for Pax7⁺, control vs. Itgb1^{-/-} (−FGF). (g) Representative images (n=25) of YFP⁺ SCs cultured for 36 h with or without FGF-2 were stained for pp38; polarized, open arrowheads; non-polarized, dashed outline; Scale bar, 10 μm. (h) Percentages of YFP⁺ SCs with polarized pp38⁺ from data in g; numerical data are expressed as mean±s.d.; n=3 experiments, ≧25 myofibers per condition. Data were compared by two-way ANOVA: *P<0.05 and n.s., not significant.

FIG. 4. Activating β1-integrin in aged SCs can rescue aged-associated SC defects. (a,b) Myofiber-associated young and aged SCs stained for Pax7, activated β1-integrin (act. β1), and DAPI 1 h after isolation; basal surface, dashed line; act. β1 patterns scored as basal (open arrowhead) or unevenly or non-detectable (non-basal) in b; Scale bar, 10 μm. All images were taken with same exposure. (b) Percentages of Pax7⁺ SCs scored by act. β1 patterns from a; n=3 experiments, ≧20 myofibers; numerical data are expressed as mean±s.d.; Student's t-test: *P<0.05. (c) Young and aged Pax7⁺ SCs stained for ILK, Parvin, Paxillin, Vinculin, and DAPI 1 h after isolation; dashed lines, basal surface; scale bar=5 μm. All images were taken with same exposure. (d) Schematic for β1-integrin activation in young (3 month) or aged (18 month) muscles after needle track injury. 2 d post injury, IgG vehicle (10 μg/ml; V), TS2/16 activating antibody (10 μg/ml; A), or RGD peptide inhibitor (10 μg/m; I) were injected into the injury site. Muscles were harvested 3 d later. (e) Muscle sections were stained for H&E or eMyHC; Scale bar, 150 μm. (f) Average number of eMyHC⁺ fibers in injured areas of each group; data represent mean±s.d.; n=3; ten sections per animal; two-way ANOVA: **P<0.01 and n.s., not significant.

FIG. 5. Activating β1-integrin in aged SCs enhances FGF signaling to promote SC expansion. (a-c) Myofiber-associated YFP⁺ aged SCs were cultured with IgG or TS2/16 (at 10 μg/ml) and with or without FGF-2 (FGF, 10 ng/ml) for 72 h, and stained for Pax7 and MyoD. (a) Images for IgG alone and TS2/16+FGF-2 treated cultures; asterisk, Pax7⁺MyoD⁺ cells; open arrowhead, MyoD⁺ cells; Scale bar, 25 μm. (b) Percentages of Pax7+MyoD⁺ versus MyoD⁺ cells in all four groups; mean±s.d.; n=3 experiments, ≧30 myofibers each; two-way ANOVA: *P<0.05 for TS2/16+FGF-2 versus others, and not significant (n.s.) between the other groups. (c) Average number of YFP⁺ cells per myofiber of the same samples in a; mean±s.d.; n=3 animals, ≧15 myofibers each two-way ANOVA: **P<0.01 for TS2/16+FGF-2 versus others, and n.s. between the other groups. (d,e) Myofiber-associated aged SCs cultured for 24 h with IgG or TS2/16 and stained for Pax7 and FGFR1. (d) Representative images (n=20); Scale bar, 10 μm. (e) Percentage of FGFR1⁺ cells within the Pax7⁺ population; mean±s.d., n=3 experiments, ≧20 myofibers per condition; Student's t-test: *P<0.05. (f,g) Aged SCs cultured with IgG or TS2/16, and with or without FGF-2, were stained for phospho-FGFR (pFGFR) and Pax7. (f) Representative images of IgG+FGF-2 and TS2/16+FGF-2 (n=20 images of each group); Scale bar, 10 μm. (g) Percentage of pFGF⁺ cells within the Pax7⁺ SC population; mean±s.d.; n=3 experiments, ≧20 myofibers scored per condition; Student's t-test: *P<0.05, ***P<0.001. (h) Reciprocal co-immunoprecipitation (co-IP) between HA-tagged Itgb1 and Flag-tagged FGFR1 in HEK293T cells, with IgG or TS2/16 added, followed by Western blot; input lysates, top two rows; IPed fractions, bottom two rows; antibodies for IP, labeled at bottom, and for Western blots, left. Open arrowheads indicate co-IPed Flag-FGFR1 (left) and HA-Itgb1 (right).

FIG. 6. Activating β1-integrin in mdx mice ameliorates dystrophic pathology and restores muscle strength. (a) Schematic of short-term IgG (V) and TS2/16 (A) treatment of 3 month old mdx mice and images of EdU⁺ myogenic nuclei that are Pax7⁺, MyoD⁺ (open arrowheads), or centrally located (asterisk). (b) Number of EdU nuclei per field (0.228 μm²); n=3 animals per treatment; ten sections per animal. (c) Schematic of long-term IgG or TS2/16 treatment and images of H&E stained muscle sections of treated mdx mice at 28 d; Scale bar, 25 μm. (d) Cross-sectional area (CSA) and (e) diameter of myofbers from data in c; n=3 animals per treatment, ten sections per animal; mean±s.d.; Student's t-test: *P<0.05, **P<0.01. (f) CSA of TA muscles from 4 groups of 3 month old mice: C57BL/10 (BL10, untreated), mdx (untreated), mdx+IgG (IgG treated), mdx+TS2/16 (TS2/16-treated); the latter two groups were treated by regimen in d. (g-j) Contractile properties of TA muscles were measured in situ: (g) Representative traces (n=5 per group) of normalized specific twitch force (sPt) and quantifications; orange vertical line, time of stimulation. (h) Representative traces (n=5 per group) for specific maximum tetanic forces (sPo) and quantifications; orange bar, duration of stimulation (300 ms). (i) Normalized tetanic force to stimulation frequency relationship. (j) Fatigue traces (left) over 180 s and fatigue indices (FI=tetanic forceₜ₀/tetanic forceₜ₁₈₀) for all groups. Numerical data are expressed as mean±s.d.; Student's t-test: *P<0.05, **P<0.01, and n.s., not significant.

FIG. 7. β1-integrin is specifically lost from Pax7+SCs. (a) Tmx regimen and assay scheme for FIG. 1a; vertical lines indicate daily intervals. (b) SCs of control (Pac7CE/+; R26RYFP/YFP) animals stained for YFP and Pax7; arrowhead, YFP+Pax7+SC; scale bar=50 μm. (c) Percentage of YFP+Pax7+ in total Pax7+ SCs; n=3 animals, ten sections per animal. Efficiency of tmx-induced YFP+SC cell marking (95%) is comparable to that using the R26RLacZ reporter15. (d) Western blot of

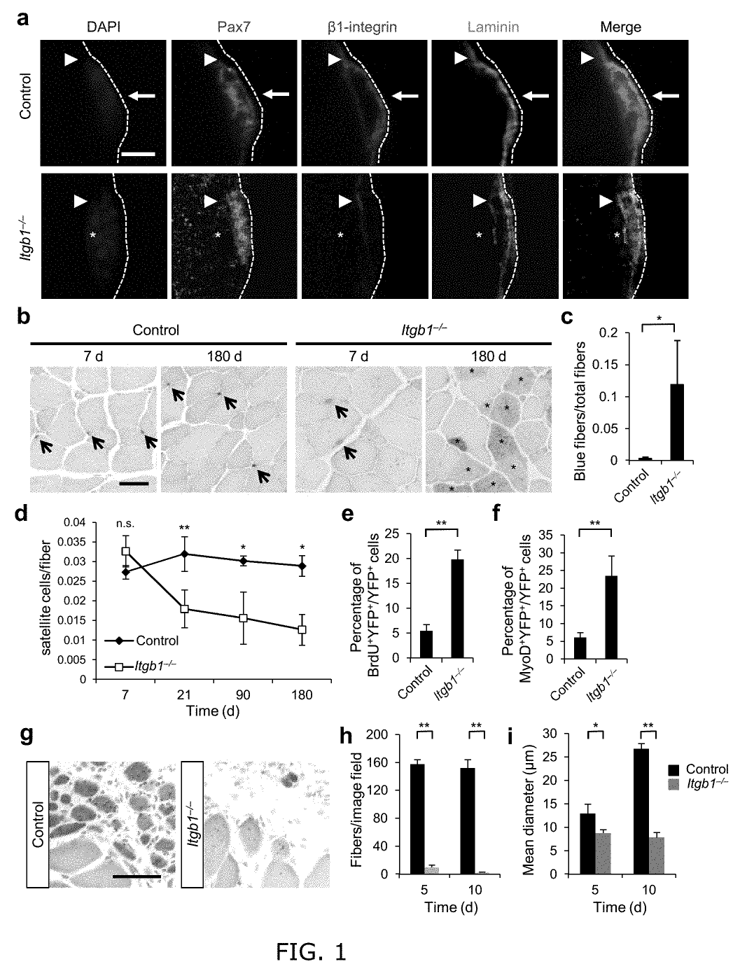

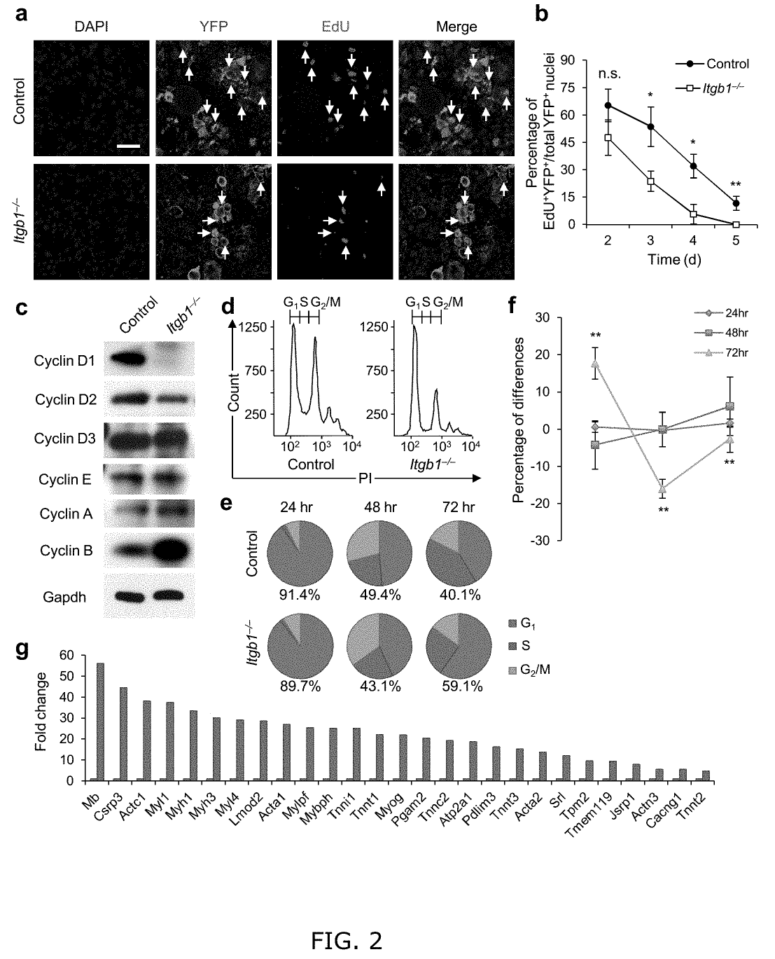

FACS isolated control and Itgb1−/− YFP+ SCs (as in FIG. 2c). Two forms of β1-integrin in control are detected; the lower band is presumably β1D-integrin. Molecular weight (Mw, in kDa) is indicated. (e) YFP+(arrows) control and Itgb1−/− SCs in vivo also show removal of β1-integrin in the mutant cell; scale bar=20 μm. Due to antibody cross-reactivity, laminin staining is not provided here; it is presented in FIG. 1a. (f) Control and Itgb1−/− myoblasts in growth media for 3 d, then without or with 1 mM staurosporine treatment for 3 h, and probed for cleaved caspase-3 immune-reactivity for PCD; scale bar=50 rm. Using this antibody in vivo, lineage marked mutant cells undergoing PCD were not found. While PCD cannot be formally excluded as a partial mechanism for mutant SC reduction, it is not believed to be a major contributor.

FIG. 8. Itgb1−/− cells show defective polarity after 30 d. (a-c) M− cadhein is at the apical side (away from laminin) in YFP+ control (a), but abnormal in YFP+ Itgb1−/− cells (b) as quantified in (c); arrows, apical side; arrowhead, basal side. Enlarged images of the insets are below (a) and (b); additional examples of mutant cells are to the right; Scale bars, 5 μm. (c) Percent control and Itgb1−/− cells with apical m− cadeherin; n=3 animals for each group, 5 sections per animal. (d) Two control SCs on myofiber stained for Par3, YFP and DAPI 30 h after isolation, show heterogeneity; enlarged images of the inset are below. (e) More examples of single control and mutant SCs stained as in (d); arrowheads, polarized Par3; Scale bars, 10 μm in (d-e). (f) Percent control and Itgb1−/− cells with polarized Par3. More than 20 fibers and 50 cells were scored for each group; Student's t-test: *P<0.05.

FIG. 9. Itgb1−/− mice are defective in the entire muscle regeneration process. (a) The injury and regeneration assay scheme for FIG. 1g-i. (b) Control TA muscle at 30 d post injury by H&E stains at low (left) and high (right 2 images, boxed areas in the left image) magnifications; arrows indicate uninjured boundary. (c) Low (left) and high (middle, boxed area in the left image) magnifications of H&E stained Itgb1−/− muscle at 30 d after injury; arrows indicate the lack of central nuclei myofibers at the uninjured boundary. The right image is trichrome stain of a nearby section revealing extensive fibrosis (green area) next to uninjured muscle fibers (dark red). n=3 for each group. Scale bars=1 mm in left 2 images and =100 μm in the high magnification images. (d) Cross sectional areas (CSA) of regenerated fibers of control and Itgb1−/−30 d post injury. (e-g) Itgb1−/− cells are perpetuated by fusion and migration defects. (e, f) Control and Itgb1−/− YFP+ SCs were cultured in differentiation media for 3 d, and stained with MHC and DAPI in (f) to determine fusion index (h); scale bar=50 μm. (g) Control and Itgb1−/− myoblasts were monitored live to measure migration velocities; numerical data=mean±s.d., n=3 experiments; Student's t-test; **P<0.01.

FIG. 10. RNA-seq data reveal that Itgb1−/− SCs display gene expression changes progressively over 72 h. Tables (a, c) and pie charts (b) summarize results from RNA-seq analyses for FIG. 2g, comparing gene expression changes in mutant vs. control cells after culture in 10% horse serum growth media for 24, 48, and 72 h. (a) Differentially expressed genes were determined by Cuffdiff 2 to have significant q-values. (b) Schematic representation of (a). Functional categories provided by PANTHER. Similar gene categories represented between 48 and 72 h, although in different numbers. Pathway analyses did not uncover significant changes in relevant signaling pathways. (c) Top 10 Upregulated and Downregulated genes as determined by Cuffdiff 2, an algorithm that estimates expression at transcript-level resolution and controls for variability evident across replicate libraries. Genes were included only if one (control or Itgb1) had FPKM≧5 to control for elevated fold changes of genes with minimal expression.

FIG. 11. Itgb1−/− SCs and control SCs treated with RGD peptide are prone to differentiation. (a, b) are support data for FIG. 2a: (a) Average number of MyoD+YFP+ cells per field (0.228 μm²) from the same samples in FIG. 2a through the course of regeneration. (b) Ratios of MyoD+ versus Pax7+ cells (in FIG. 2b). Data are expressed as mean±s.e.m; Student's t-test: *P<0.05; **P<0.01; n=3 animals per time point, 10 sections scored per animal. (c) Myofiber-associated control young YFP+ SCs of were cultured for 48 h with scrambled or RGD peptide and with or without FGF-2 and stained for Pax7 and MyoD. (d) SCs are self-renewed (Pax7+MyoD−), proliferating and self-renewable (Pax7+MyoD+; asterisk), or committed to differentiation (Pax7-MyoD+; open arrowhead); confocal images; scale bar=10 μm. (e) Pie charts summarize data in (a); n≧25 myofibers per condition; two-way ANOVA was used for paired comparison: P<0.05, scrambled vs. scrambled+FGF-2 for Pax7-MyoD+ and scrambled vs. RGD+FGF-2; not significant, RGD vs. RGD+FGF-2 for Pax7-MyoD+. RGD-peptide treatment caused SCs to fall off the myofiber over time, and therefore shorter time frame was used. In this context, there were very few Pax7+MyoD− cells. Our conclusion is based on Pax7+MyoD+ fractions, as these cells have the potential to self-renew by turning off MyoD.

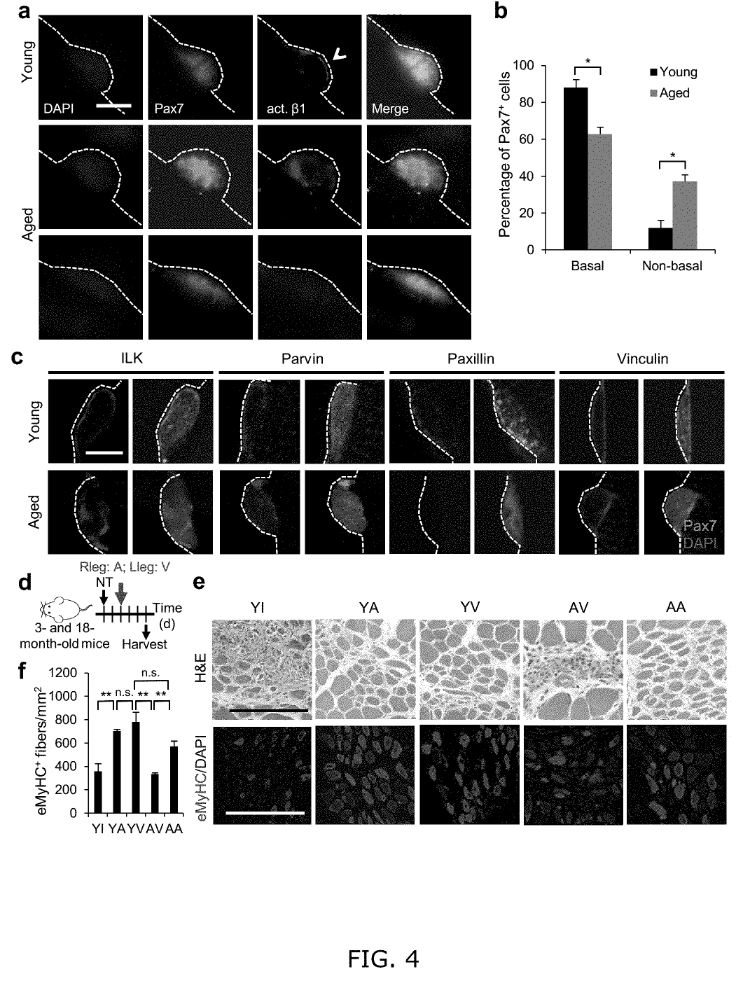

FIG. 12. Distribution of β1-integrin in aged and dystrophic SCs. (a) Aged SCs show laminar-localized pan β1-integrin. YFP+ myofiber-associated SCs of aged mice at 1 h after isolation stained for pan β1-integrin. Localization pattern mirrors young control SCs stained for pan-β1 integrin (FIG. 1a); dashed lines outline the basal side of the myofiber; scale bar=10 μm. (b, c) SCs on mdx myofibers have dysregulated β1-integrin activity: (b) Pax7+ SCs on myofibers after isolation from mdx mice show basally restricted (top) and non-basally restricted (middle and bottom) activated β1-integrin (act. β1) patterns (same exposure time), compared to control (FIG. 4a, b, young SC). (c) Percentages of act. β1 displaying basal and non-basal pattern in the Pax7+SC population.

FIG. 13. TS2/16 specifically activates β1-integrin and support long-term muscle regeneration. (a-c) TS2/16 does not rescue the regeneration deficiency of Itgb1−/− muscle: (a) Tmx regimen, CTX injury, and TS2/16 injection scheme. 2-4 d post injury, 6×10 μl injections into the injury site of either vehicle IgG or TS2/16 (10 μg/ml). (b) TA muscles were harvested at 5 d post injury, and cross-sections were H&E stained; scale bar=150 μm. (c) Regeneration was quantified by central nuclei myofibers in the injured area. Data represent mean±s.e.m.; n=3 animals per condition; ten sections per animal; one-way ANOVA: n.s., not significant. (d) TS2/16 activates FAK phosphorylation (pFAK) in control but not Itgb1−/− myoblasts. (e-g) TS2/16-enhanced aged muscle regeneration persists to 30 d post injury: (e) Representative images for IgG-treated (top) and TS2/16-treated (bottom) aged muscle sections 30 d after needle-track injury; arrows indicate boundaries of regenerative tracks; n=4 animals for each group; scale bar=100 μm. (f, g) Average number of fibers (f) with centrally located nuclei per regenerative area (0.216 mm2) and average fiber diameters (g) of each group are presented as mean±sem; Student's t test: *P<0.05.

FIG. 14. TS2/16 and FGF-2 increase the fraction of aged SCs displaying polarized pp38. (a) Two cells on a single myofiber show heterogeneity of pp38 staining between YFP-marked SCs for FIG. 2g. The cell in the inset shows polarized pp38, while the other shows none-to-minimal pp38 signal; scale bars=10 μm. (b) Additional single cell examples of various patterns of pp38 distribution in aged myofiber-associated lineage-marked YFP+ SCs cultured for 30 h with control IgG or TS2/16 (10 μg/ml), with or without FGF-2 (10 ng/ml); scale bar=10 μm. (c) Percentages of SCs with polarized pp38 (arrowhead in a and b); those with only a few puncta of pp38 signal were not counted as polarized; n=3 experimental replicates, ≧25 myofibers each condition per replicate; Student's t-test paired comparison: *P<0.05, **P<0.01.

FIG. 15. TS2/16-treated mdx mice have reduced fraction of SCs outside the myofiber basal lamina. (a) control C57/BL10 and (b) mdx TA muscles stained for Pax7, laminin, pan-β1-integrin, and DAPI; arrows, Pax7+ SCs inside the myofiber laminin basal lamina (BL); arrowheads, Pax7+ SCs outside the BL. (c) Percentages of Pax7+ SCs outside the BL for all groups in (a, b, d, and e); n=3 animals per group; >100 SCs counted per group; Student's t-test, paired comparison: *P<0.05, **P<0.01, and n.s., not significant. (d) IgG-treated mdx and (e) TS2/16-treated mdx TA muscles stained and labeled as (a) and (b), and quantified in (c). Enlarged images for each inset in (a, b, d, and e) are to the left. (e) has two insets; images for the top inset is to the immediate right, and for the bottom inset, further right. Scale bars, 10 μm.

FIG. 16. Models for β1-integrin function in young and aged SC niches. Keys to the symbols are to the right. Top panel: Young SCs uses β1-integrin to sense and occupy the quiescent niche, and they can support injury-induced expansion and renewal. Middle panel: β1-integrin mutant young SCs are prone to loss, and cannot support injury-induced expansion and renewal. FGF-2 partially rescues these defects in vitro, but unlikely to rescue the fusion defect to support regeneration in vivo. Bottom panel: During the aging process, SCs with sufficient overall integrin activity cooperate with increasing levels of FGF-2, break quiescence, and become lost. The remaining aged SCs with integrin dystruglation, reflected by abnormal patterns of active β1-integrin (β1-integrin*), are non-responsive to FGF-2, but can be rescued by TS2/16 and FGF-2.

### DETAILED DESCRIPTION

Sarcopenia, the slow progressive loss of skeletal muscle mass concomitant with advancing age, affects elderly people. Age-related muscle wasting is characterized by loss of muscle quantity and quality due to changes in muscle metabolism, function and regeneration¹. The poor regeneration of aged muscle is not attributed solely to the loss of stem cells (i.e. satellite cells, SCs)^{2,3}, although SC numbers decline during aging in mice and humans. Instead, the impaired regeneration is linked to aged-related changes in both extrinsic systemic and local environments as well as intrinsic defects⁴⁻¹¹.

A well-studied niche signal, fibroblast growth factor-2 (FGF-2), has important roles in driving SC proliferation. SCs are maintained in a quiescent state by repressing FGF-2 signals⁷. Aging increases the level of FGF-2 in skeletal muscle and decreases the level of FGF signaling inhibitor Spryl in SCs, which results in loss of a portion of the SC pool due to breaking quiescence². Paradoxically, aged SCs are non-responsive to FGF-2 during their proliferation and self-renewal⁹. Inhibiting FGF signaling in aged SCs rescues the quiescent phenotype at the expense of regeneration², whereas ectopically activating FGF receptor 1 (FGFR1) rescues the proliferative capacity of aged SCs⁹. It is unclear what underlies the differential requirement for FGF and what causes the desensitization of FGFR1, but changes in interactions with other cell surface proteins may contribute.

Integrins adhere to the extra cellular matrix (ECM) to cooperate with different growth factor signaling pathways depending on the cell type and context^{12,13}. Integrins are heterodimers comprised of an a and a 1 chain that function to link the ECM to the actin cytoskeleton¹⁴. There are 18α and eight β chains, which can form at least 28 isoforms. Of particular relevance to skeletal muscles are α7 and α5 integrins and laminin α2 as their mutations cause muscular dystrophies^{14,15}. Inactivation of Itgb1 (encoding β1-integrin) in the embryonic muscle lineage causes defects in muscle cell migration, fusion, and sarcomere assembly, but not progenitor proliferation¹⁶. Although β1-integrin is conspicuously expressed by adult SCs, its role has not been examined, especially with respect to modulating FGF signaling. The present disclosure demonstrates that β1-integrin is the sensor of the SC niche that maintains quiescence of SCs during homeostasis and it also cooperates with FGF signaling to promote SC proliferation and renewal after injury. Activating β1-integrin signaling restores FGF sensitivity in aged SCs and improves muscle regeneration. Activating β1-integrin in the mdx mouse¹⁷, a model for Duchenne muscular dystrophy, can also promote SC expansion and improve function. β1-integrin is a potential therapeutic target of pathological conditions in which the SC niche is compromised.

The presently disclosed technology provides methods of treating or preventing a skeletal muscle condition or disease, especially relating to decreased or reduced satellite cell proliferation, in a subject in need thereof, comprising administering to the subject an effective amount of a composition or treatment which increases the level or amount of activated β1-integrin in the subject, especially in the microenvironment of the niche, wherein the composition or treatment increases the frequency or number of the subject's skeletal muscle stem cells or satellite cells preferably in the microenvironment of the niche, and/or increases the size of regenerating myofibers of the subject, and/or increases the efficiency of myogenic colony formation of the subject, thereby promoting skeletal muscle regeneration in the subject.

The presently disclosed technology provides methods of treating or preventing sarcopenia in a subject in need thereof, comprising administering to the subject an effective amount of a composition or treatment which increases the level of activated β1-integrin in the subject, especially in the microenvironment of the niche, wherein the composition or treatment increases the frequency or number of the subject's skeletal muscle stem cells or satellite cells, and/or increases the size of regenerating myofibers of the subject, and/or increases the efficiency of myogenic colony formation of the subject, thereby treating or preventing sarcopenia in the subject.

Skeletal muscle conditions or diseases which may be treated according to the presently disclosed technology include any of atrophy, bone fractures associated with muscle wasting or weakness, cachexia, muscular dystrophy, diabetes, exercise induced skeletal muscle fatigue, fatigue, frailty, inflammatory myopathies, metabolic syndromes, neuromuscular disease, obesity, post-surgical muscle weakness, post-traumatic muscle weakness, sarcopenia, toxin exposure, wasting, weakness and/or combinations thereof.

The presently disclosed technology provides methods of treating or decreasing or reducing the severity of skeletal muscle conditions, such as atrophy, bony fractures associated with muscle wasting or weakness, cachexia, muscular dystrophy, diabetes, exercise-induced skeletal muscle fatigue, fatigue, frailty, inflammatory myositis, metabolic syndrome, neuromuscular disease, obesity, post-surgical muscle weakness, post-traumatic muscle weakness, sarcopenia, toxin exposure, wasting, and weakness and/or combination thereof.

Frailty is a syndrome characterized by meeting at least one of the following five attributes: unintentional weight loss, muscle weakness, slow walking speed, exhaustion, and low physical activity. Cachexia is characterized as a state often associated with cancer or other serious diseases or conditions, (e.g., chronic obstructive pulmonary disease, chronic kidney disease), that is characterized by progressive weight loss, muscle atrophy and fatigue, due to the deletion of adipose tissue and skeletal muscle. Post-surgical muscle weakness refers to a reduction in the strength of one or more muscles following surgical procedure. Weakness may be generalized (i.e., total body weakness) or localized to a specific area, side of the body, limb, or muscle. Post-traumatic muscle weakness is characterized by a reduction in the strength of one or more muscles following a traumatic episode (e.g., bodily injury). Neuromuscular disease includes any disease or condition that affects any part of the nerve and muscle. Neuromuscular disease encompasses critical illness polyneuropathy, prolonged neuromuscular blockade, acute myopathy as well as acute inflammatory demyelinating polyradiculoneuropathy, amyotrophic lateral sclerosis (ALS), autonomic neuropathy, Charcot-Marie-Tooth disease and other hereditary motor and sensory neuropathies, chronic inflammatory demyelinating polyradiculoneuropathy, dermatomyositis/polymyositis, diabetic neuropathy, dystrophinopathies, endocrine myopathies, focal muscular atrophies, hemifacial spasm, hereditary neuropathies of the Charcot-Marie-Tooth disease type, inclusion body myositis, Kennedy disease, Lambert-Eaton myasthenic syndrome, muscular dystrophy (e.g., limb-girdle, Duchenne, Becker, myotonic, facioscapulohumeral, etc.), metabolic myopathies, metabolic neuropathy, multifocal motor neuropathy with conduction blocks, myasthenia gravis, neuropathy of Friedreich Ataxia, neuropathy of leprosy, nutritional neuropathy, periodic paralyses, primary lateral sclerosis, restrictive lung disease, sarcoidosis and neuropathy, Schwartz-Jampel Syndrome, spinal muscular atrophy (SMA), stiff person syndrome, thyroid disease, traumatic peripheral nerve lesions, vasculitic neuropathy, among others. Sarcopenia refers to a loss of skeletal muscle mass, quality, and strength. Often sarcopenia is associated with aging, but may also occur in association with HIV infection and a variety of chronic conditions. Sarcopenia may lead to frailty, for example, in the elderly. Sacropenia also encompasses a condition or symptom associated with sacropenia including, but not limited to loss of skeletal muscle mass, muscle weakness, fatigue, disability, and morbidity.

A skeletal muscle condition due to aging may refer to a skeletal muscle condition described herein which is attributable to a subject's age. The subject may be one who is at risk of developing a skeletal muscle condition due to aging. In some embodiments, the subject treated by methods of the presently disclosure may be an elderly subject, such as a person over the age of 45, 50, 55, 60, 65, 70, 75, 80, 85, 90, or 100 years old.

Methods of the presently disclosed technology may be used to treat a subject, which includes human and non-human animals, such as a vertebrate—including a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In certain embodiments, the subject is a mammal, e.g., a primate, e.g., a human.

The presently disclosed technology provides methods of increasing the efficiency or robustness of muscle repair in a subject, such as methods of increasing or accelerating the recovery from muscle damage in a subject.

The presently disclosed technology provides methods of increasing the strength (e.g., muscle strength) or exercise endurance capacity (e.g., muscle endurance) in a subject in need thereof, comprising administering to the subject an effective amount of a composition or treatment which increases the level of activated β1-integrin in the subject, thereby increasing the strength or exercise endurance capacity.

Compositions of the presently disclosed technology include a polypeptide, such as an antibody, including a human or humanized versions, chimeric or functional fragments or variants thereof, and/or RGD-containing peptides, such as fibronectin and fragments and/or variants thereof, that activate β1-integrin.

Antibodies, human or humanized antibodies, chimeric or functional fragments or variants thereof of the present disclosure, which specifically binds and/or activates integrin β1 may include one, two, three, four, five, or all six of the CDRs or CDR combinations as described herein.

Examples of antibody functional fragments include, but are not limited to, complete antibody molecules, antibody fragments, such as Fv, single chain Fv (scFv), complementarity determining regions (CDRs), VL (light chain variable region), VH (heavy chain variable region), Fab, F(ab)2′ and any combination of those or any other functional portion of an immunoglobulin peptide capable of binding to target antigen (see, e.g., Fundamental Immunology (Paul ed., 3d ed. 1993). As appreciated by one of skill in the art, various antibody fragments can be obtained by a variety of methods, for example, digestion of an intact antibody with an enzyme, such as pepsin; or de novo synthesis. Antibody fragments are often synthesized de novo either chemically or by using recombinant DNA methodology. Antibody and antibody fragments may be produced by the modification of whole antibodies, or those synthesized de novo using recombinant DNA methodologies (e.g., single chain Fv) or those identified using phage display libraries. Antibodies may include bivalent or bispecific molecules, diabodies, triabodies, and tetrabodies.

Immunoglobulin heavy chains may be referred to as VH and may include an Fv, scFv, a disulfilde-stabilized Fv (dsFv) or Fab. Immunoglobulin light chains may be referred to as VL and include of an Fv, scFv, dsFv or Fab.

Single chain Fv or scFv are an antibody in which the variable domains of the heavy chain and of the light chain of a traditional two chain antibody have been joined to form one chain. Typically, a linker peptide is inserted between the two chains to allow for the stabilization of the variable domains without interfering with the proper folding and creation of an active binding site. A single chain humanized antibody of the present disclosure, e.g., humanized anti-integrin β1 antibody, may bind as a monomer. Other exemplary single chain antibodies may form diabodies, triabodies, and tetrabodies. (See, e.g., Hollinger et al., 1993). Further the humanized antibodies of the present disclosure, e.g., humanized anti-integrin β1 antibody may also form one component of a reconstituted antibody or antibody fragment, e.g., a Fab, a Fab′ monomer, a F(ab)′2 dimer, or an whole immunoglobulin molecule. Thus, a humanized antibody of the present disclosure may further contain a human Fc region.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a VH CDR3 is located in the variable domain of the heavy chain of the antibody in which it is found, whereas a VL CDR1 is the CDR1 from the variable domain of the light chain of the antibody in which it is found. The numbering of the light and heavy chain variable regions described herein is in accordance with Kabat (see, e.g., Johnson et al., (2001) “Kabat Database and its applications: future directions” Nucleic Acids Research, 29: 205-206; and the Kabat Database of Sequences of Proteins of Immunological Interest, Feb. 22, 2002 Dataset).

The positions of the CDRs and framework regions can be determined using various well known definitions in the art, e.g., Kabat, Chothia, international ImMunoGeneTics database (IMGT), and AbM (see, e.g., Johnson et al., supra; Chothia & Lesk, 1987, Canonical structures for the hypervariable regions of immunoglobulins. J. Mol. Biol. 196, 901-917; Chothia C. et al., 1989, Conformations of immunoglobulin hypervariable regions. Nature 342, 877-883; Chothia C. et al., 1992, structural repertoire of the human VH segments J. Mol. Biol. 227, 799-817; Al-Lazikani et al., J. Mol. Biol. 1997, 273(4)). Definitions of antigen combining sites are also described in the following: Ruiz et al., IMGT, the international ImMunoGeneTics database. Nucleic Acids Res., 28, 219-221 (2000); and Lefranc, M.-P. IMGT, the international ImMunoGeneTics database. Nucleic Acids Res. January 1; 29(1):207-9 (2001); MacCallum et al, Antibody-antigen interactions: Contact analysis and binding site topography, J. Mol. Biol., 262 (5), 732-745 (1996); and Martin et al, Proc. Natl. Acad. Sci. USA, 86, 9268-9272 (1989); Martin, et al, Methods Enzymol., 203, 121-153, (1991); Pedersen et al, Immunomethods, 1, 126, (1992); and Rees et al, In Sternberg M. J. E. (ed.), Protein Structure Prediction. Oxford University Press, Oxford, 141-172 1996).

A humanized antibody refers to an antibody that comprises a donor antibody binding specificity, i.e., the CDR regions of a donor antibody, such as a mouse, bovine, canine, equine, monkey, mustelid, porcine, primate, rabbit, rat, or sheep, monoclonal antibody, grafted onto human framework sequences. A humanized antibody may bind to the same epitope as the donor antibody and typically has at least 25% of the binding affinity. Assays for binding affinity are known in the art. Methods to determine whether the antibody binds to the same epitope are well known in the art, see, e.g., Harlow & Lane, Using Antibodies, A Laboratory Manual, Cold Spring Harbor Laboratory Press, 1999, which discloses techniques to epitope mapping or alternatively, competition experiments, to determine whether an antibody binds to the same epitope as the donor antibody.

Compositions of the presently disclosed technology include a polypeptide, such as an antibody, including human or humanized versions, chimeric or functional fragments or variant thereof, that activate β1-integrin, in a manner similar to TS2/16, which binds to amino acids 207-218 of the β1 chain primary structure, such as antibodies 8A2 and AIA5 (Tsuchida et al. “Classification of “activation” antibodies against integrin β chain” FEBS Letters 416 (1997) 212-216).

Human β1-integrin is coded for by Itgb1 and has the following amino acid structure (ACCESSION: P05556, gi|218563324|) and is also identified as CD29 (SEQ ID NO:1):

MNLQPIFWIG LISSVCCVFA QTDENRCLKA NAKSCGECIQ
AGPNCGWCTN STFLQEGMPT SARCDDLEAL KKKGCPPDDI
ENPRGSKDIK KNKNVTNRSK GTAEKLKPED ITQIQPQQLV
LRLRSGEPQT FTLKFKRAED YPIDLYYLMD LSYSMKDDLE
NVKSLGTDLM NEMRRITSDF RIGFGSFVEK TVMPYISTTP
AKLRNPCTSE QNCTSPFSYK NVLSLTNKGE VFNELVGKQR
ISGNLDSPEG GFDAIMQVAV CGSLIGWRNV TRLLVFSTDA
GFHFAGDGKL GGIVLPNDGQ CHLENNMYTM SHYYDYPSIA
HLVQKLSENN IQTIFAVTEE FQPVYKELKN LIPKSAVGTL
SANSSNVIQL IIDAYNSLSS EVILENGKLS EGVTISYKSY
CKNGVNGTGE NGRKCSNISI GDEVQFEISI TSNKCPKKDS
DSFKIRPLGF TEEVEVILQY ICECECQSEG IPESPKCHEG
NGTFECGACR CNEGRVGRHC ECSTDEVNSE DMDAYCRKEN
SSEICSNNGE CVCGQCVCRK RDNTNEIYSG KFCECDNFNC
DRSNGLICGG NGVCKCRVCE CNPNYTGSAC DCSLDTSTCE
ASNGQICNGR GICECGVCKC TDPKFQGQTC EMCQTCLGVC
AEHKECVQCR AFNKGEKKDT CTQECSYFNI TKVESRDKLP
QPVQPDPVSH CKEKDVDDCW FYFTYSVNGN NEVMVHVVEN
PECPTGPDII PIVAGVVAGI VLIGLALLLI WKLLMIIHDR
REFAKFEKEK MNAKWDTGEN PIYKSAVTTV VNPKYEGK

Isoforms and splice variants of the human β1-integrin also exist and are included herein as therapeutic targets for pathological conditions of the muscle in which the stem cell niche is compromised.

Further, the presently disclosed technology includes alternative methods of targeting all integrins relevant to SC expansion and FGF-sensitivity, e.g., RGD-binding integrins, including the following integrins: α₃β₁, α₅β₁, α₈β₁, α_{V}β₁, α_{V}β₃, α_{V}β₅, α_{V}β₆, α_{IIb}β₃. Moreover, fibronectin, fragments of fibronectin and/or other RGD-containing ECM components may be useful in activating β1-integrins according to the presently disclosed technology and therefore may be useful in causing or facilitating any one or more of increasing the frequency or number of a subject's skeletal muscle stem cells or satellite cells, increasing the size of regenerating myofibers, and/or increasing the efficiency of myogenic colony formation, thereby rejuvenating the skeletal muscle stem cells or satellite cells in the subject.

Compositions of the presently disclosed technology include a polypeptide, such as an antibody, including a human or humanized versions, chimeric or functional fragments or variants thereof, that activate β1-integrin, such as antibodies produced from hybridomas TS2/16, 8A2 and AIA5 (Tsuchida et al. “Classification of “activation” antibodies against integrin 1 chain” FEBS Letters 416 (1997) 212-216, and Takada et al “Identification of a regulatory region of integrin beta 1 subunit using activating and inhibiting antibodies” J Biol Chem. 1993 Aug. 15; 268(23):17597-601), as well as ATCC deposit HB-243 (HB-243) and ECACC deposit 93070777, or human or humanized versions, chimeric or functional fragments or variants thereof, that activate β1-integrin.

Sequences of antibodies TS2/16, 8A2, AIA5, HB-243 and ECACC 93070777 may be obtained by means known in the art, such as is described in “Rapid Cloning of Antibody Variable Regions Using SMART Technology” Tech Note, Takara Clontech (2016) (http://www.clontech.com/US/Products/cDNA_Synthesis_and_Library_Construction/RACE_Rapid_Amplification_of_cDNA_Ends/Cloning_Antib ody_Variable_Regions) or “Antibody Protein Sequencing” Rapid Novor (2016) (http://www.rapidnovor.com/antibody/).

Human or humanized antibodies, chimeric or functional fragments or variants thereof of the present disclosure, which specifically binds and/or activates integrin β1 may include a heavy chain variable (VH) region and a light chain variable (VL) region or portions or fragments thereof, wherein the VH region has greater than about 95%, 96%, 97%, 98% or 99% identity to a VH region of an antibody selected from TS2/16, 8A2, AIA5, HB-243 or ECACC 93070777 or at least 1, 2 or 3 complementarity-determining regions (CDRs) thereof; and/or wherein the VL region has greater than about 95%, 96%, 97%, 98% or 99% identity to a VL region of an antibody selected from TS2/16, 8A2, AIA5, HB-243 or ECACC 93070777, or at least 1, 2 or 3 complementarity-determining regions (CDRs) thereof.

Humanized antibodies described herein can be produced using a variety of techniques known in the art, including, but not limited to, CDR-grafting (see e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see, e.g., European Patent Nos. EP 592,106 and EP 519,596; Padlan, 1991, Molecular Immunology 28(4/5):489-498; Studnicka et al., 1994, Protein Engineering, 7(6):805-814; and Roguska et al., 1994, Proc. Natl. Acad. Sci., 91:969-973), chain shuffling (see, e.g., U.S. Pat. No. 5,565,332), and techniques disclosed in, e.g., U.S. Pat. No. 6,407,213, U.S. Pat. No. 5,766,886, International Publication No. WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16): 10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mal. Biol., 235 (3):959-73 (1994). Often, framework (FW) residues in the FW regions will be substituted with the corresponding residue from the CDR donor antibody to alter, preferably improve, antigen binding. These FW substitutions are identified by methods well known in the art, e.g., by modeling of the interactions of the CDR and FW residues to identify FW residues important for antigen binding and sequence comparison to identify unusual FW residues at particular positions. (See, e.g., Queen et al., U.S. Pat. No. 5,585,089; and Riechmann et al., 1988, Nature, 332:323, which are incorporated herein by reference in their entireties.)

A humanized anti-CD29 antibody has one or more amino acid residues introduced into it from a source which is nonhuman. These nonhuman amino acid residues are often referred to as “import” residues, which are typically taken from an “import” variable domain. Thus, humanized antibodies comprise one or more CDRs from nonhuman immunoglobulin molecules and framework regions from human. Humanization of antibodies is well-known in the art and can essentially be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CD Rs or CDR sequences for the corresponding sequences of a human antibody, i.e., CDR-grafting (EP 239,400; PCT Publication No. WO 91/09967; and U.S. Pat. Nos. 4,816,567; 6,331,415; 5,225,539; 5,530,101; 5,585,089; 6,548,640). In such humanized chimeric antibodies, substantially less than an intact human variable domain has been substituted by the corresponding sequence from a nonhuman species. In practice, humanized antibodies are typically human antibodies in which some CDR residues and possibly some FW residues are substituted by residues from analogous sites in rodent antibodies. Humanization of anti-CD19 antibodies can also be achieved by veneering or resurfacing (EP 592,106; EP 519,596; Padlan, 1991, Molecular Immunology 28(4/5):489-498; Studnicka et al., Protein Engineering, 7(6): 805-814 (1994); and Roguska et al., Proc. Natl. Acad. Sci., 91:969-973 (1994)) or chain shuffling (U.S. Pat. No. 5,565,332).

The choice of human variable domains, both light and heavy, to be used in making the humanized antibodies is to reduce antigenicity. According to the so-called “bestfit” method, the sequence of the variable domain of a rodent antibody is screened against the entire library of known human variable-domain sequences. The human sequences which are most closely related to that of the rodent are then screened for the presence of specific residues that may be critical for antigen binding, appropriate structural formation and/or stability of the intended humanized mAb (Sims et al., J. Immunol., 151:2296 (1993); Chothia et al., J. Mal. Biol., 196:901 (1987). The resulting FW sequences matching the desired criteria are then used as the human donor FW regions for the humanized antibody.

Another method uses a particular FW derived from the consensus sequence of all human antibodies of a particular subgroup of light or heavy chains. The same FW may be used for several different humanized anti-CD29 antibodies (Carter et al., Proc. Natl. Acad. Sci. USA, 89:4285 (1992); Presta et al., J. Immunol., 151:2623 (1993).

Anti-CD29 antibodies can be humanized with retention of high affinity for CD 29 and other favorable biological properties. According to one aspect of the presently disclosed technology, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind CD 29 and activate β1-integrin in a manner substantially the same as TS2/16. In this way, FW residues can be selected and combined from the recipient and import sequences so that the desired antibody characteristic, for example affinity for CD29 and to activate β1-integrin in a manner substantially the same as TS2/16, is achieved. In general, the CDR residues are directly and most substantially involved in influencing antigen binding.

A humanized antibody may retain a similar antigenic specificity as the original antibody, i.e., in the presently disclosed technology, the ability to bind human CD 29 antigen and to activate β1-integrin in a manner substantially the same as TS2/16. However, using certain methods of humanization, the affinity and/or specificity of binding of the antibody for human CD 29 antigen may be altered using methods of directed evolution, as described by Wu et al., J. Mal. Biol., 294:151 (1999).

The anti-CD29 antibodies herein specifically include chimeric antibodies (immunoglobulins) in which a portion of the heavy and/or light chain is identical with or homologous to corresponding sequences in antibodies derived from a particular species or belonging to a particular antibody class or subclass, while another portion of the chain is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include “primatized” antibodies comprising variable domain antigen-binding sequences derived from a nonhuman primate (e.g., Old World Monkey, such as baboon, rhesus or cynomolgus monkey) and human constant region sequences (U.S. Pat. No. 5,693,780).

The presently disclosed technology further provides polynucleotides comprising a nucleotide sequence encoding a human, humanized, or chimeric anti-CD29 antibody of the present disclosure or fragments thereof. The presently disclosed technology also encompasses polynucleotides that hybridize under stringent or lower stringency hybridization conditions, as defined herein, to polynucleotides that encode a human, humanized, or chimeric antibody that specifically binds to human or mouse CD 29.

Stringent hybridization conditions include, but are not limited to, hybridization to filter-bound DNA in 6× sodium chloride/sodium citrate (SSC) at about 45° C. followed by one or more washes in 0.2×SSC/0.1% SDS at about 50-65° C., highly stringent conditions such as hybridization to filter-bound DNA in 6×SSC at about 45° C. followed by one or more washes in 0.1×SSC/0.2% SDS at about 60° C., or any other stringent hybridization conditions known to those skilled in the art (see, for example, Ausubel, F. M. et al., eds. 1989 Current Protocols in Molecular Biology, vol. 1, Green Publishing Associates, Inc. and John Wiley and Sons, Inc., NY at pages 6.3.1 to 6.3.6 and 2.10.3).

The polynucleotides may be obtained, and the nucleotide sequence of the polynucleotides determined, by any method known in the art. For example, if the nucleotide sequence of the antibody is known, a polynucleotide encoding the antibody may be assembled from chemically synthesized oligonucleotides (e.g., as described in Kutmeier et al., BioTechniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

A polynucleotide encoding an antibody may also be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, preferably polyA+RNA, isolated from, any tissue or cells expressing the antibody, such as hybridoma cells selected to express an antibody) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

The presently disclosed technology further provides a vector contain one or more nucleotide sequences encoding a human, humanized, or chimeric anti-CD29 antibody described herein or fragments thereof.

The presently disclosed technology further provides an isolated cell comprising a vector wherein the vector contains one or more nucleotide sequences encoding a human, humanized, or chimeric anti-CD 29 antibody of the disclosure or fragments thereof.

Chimeric, human, and humanized anti-CD29 monoclonal antibodies described herein include those of the IgGI, IgG2, IgG3, or IgG4 human isotype.

The present disclosure provides therapeutic compositions containing an effective amount of a polypeptide, such as an antibody, including a human or humanized versions, chimeric or functional fragments or variants thereof, and/or other agent(s), such as fibronectin or fragments thereof or other RGD-containing components, that activate β1-integrin and are useful for at least one of improving skeletal muscle regeneration in a population of aged satellite cells, renewing, restoring or increasing FGF-2 sensitivity of a population of aged satellite cells, and improving regeneration and recovery of skeletal muscles after injury.

Therapeutic compositions of the present disclosure will commonly include a solution of an active agent, such as a polypeptide, such as an antibody, including a human or humanized versions, chimeric or functional fragments or variants thereof, and/or other agent(s) that activate β1-integrin, according to the present disclosure dissolved in a pharmaceutically acceptable carrier, such as an aqueous carrier. A variety of aqueous carriers can be used, e.g., buffered saline. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of active agent in a formulation according to the present disclosure can vary widely, and will be selected primarily based on fluid volumes, viscosities, body weight and the like in accordance with the particular mode of administration selected and the patient's needs.

A typical pharmaceutical composition of the present disclosure for intravenous administration would be about 0.1 to 10 mg per patient per day. Dosages from 0.1 up to about 100 mg per patient per day may be used. Actual methods for preparing administrable compositions will be known or apparent to those skilled in the art and are described in more detail in such publications as REMINGTON'S PHARMACEUTICAL SCIENCE, 19TH ED., Mack Publishing Company, Easton, Pa. (1995).

The compositions of the present disclosure may be administered for therapeutic treatments. In therapeutic applications, compositions are administered to a patient in need to treatment, in an amount sufficient to treat or at least partially arrest the disease and its complications. An amount adequate to accomplish this is a therapeutically effective dose. Amounts effective for this use will depend upon the severity of the condition and the general state of the patient's health. An effective amount of the compound is that which provides either subjective relief of a symptom(s) or an objectively identifiable improvement as noted by the clinician or other qualified observer.

Single or multiple administrations of the compositions may be administered depending on the dosage and frequency as required and tolerated by the patient. In any event, the composition should provide a sufficient quantity of the proteins or active agent(s) of the present disclosure to effectively treat the patient. The dosage may be administered once or may be applied periodically until either a therapeutic result is achieved or until side effects warrant discontinuation of therapy. Generally, the dose is sufficient to treat or ameliorate symptoms or signs of disease without producing unacceptable toxicity to the patient.

Controlled release parenteral formulations of the immunoconjugate compositions of the present invention can be made as implants, oily injections, or as particulate systems. For a broad overview of protein delivery systems see, Banga, A. J., THERAPEUTIC PEPTIDES AND PROTEINS: FORMULATION, PROCESSING, AND DELIVERY SYSTEMS, Technomic Publishing Company, Inc., Lancaster, Pa., (1995). Particulate systems include microspheres, microparticles, microcapsules, nanocapsules, nanospheres, and nanoparticles. Microcapsules contain the therapeutic protein as a central core. In microspheres the therapeutic is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly. See, e.g., Kreuter, J., COLLOIDAL DRUG DELIVERY SYSTEMS, J. Kreuter, ed., Marcel Dekker, Inc., New York, N.Y., pp. 219-342 (1994); and Tice & Tabibi, TREATISE ON CONTROLLED DRUG DELIVERY, A. Kydonieus, ed., Marcel Dekker, Inc. New York, N.Y., pp. 315-339, (1992).

Without wishing to be bound to any specific mechanism, the present examples are believed to frame a model in which β1-integrin acts as a sensor of the SC niche that declines in function upon aging (FIG. 16). Because Itgb1^{-/-} cells show compromised pErk induction by FGF-2, their quiescence breaking is more likely due to cell polarity defects than overt FGF-Erk signaling². In the regenerative context, β1-integrin has a distinct role in cooperating with FGF-2 to drive SC proliferation and renewal. In aged muscle, changes in ECM^{29,30} impose physiological relevant alterations in integrin activity, which likely contribute to the decline of SC's FGF sensitivity. The current view regarding FGF and aging SCs is not clear. Aging SCs are sensitive to increased FGF-Erk signaling, causing quiescence break and loss², and yet aged SCs are insensitive to FGF-2 for expansion in vitro⁹. The presently disclosed technology suggests that a fraction of aging SCs with sufficient integrin activity cooperates with increasing FGF-2 to break quiescence and becomes lost, while those with dysregulated integrin activity insufficient to support FGF signaling, remain. As such the remaining aged SCs cannot support robust regeneration after injury, unless integrin activity is re-established, e.g. by TS2/16 in the examples of the present disclosure, to restore FGF signaling.

While activating β1-integrin alone can improve regeneration in both aged and dystrophic muscles, it may also prove effective to target all integrins relevant to SC expansion and FGF-sensitivity, e.g. RGD-binding integrins. There are minimally eight integrins (three of which contain β1-integrin) that bind to the RGD motif, which is the integrin binding site of many ECM molecules, including fibronectin¹⁴. While β1-integrin and fibronectin cooperate with FGF-2, contributions by other RGD-containing ECM components cannot be excluded. Conversely, as SCs express almost all α- and β-integrins²⁶, defining the contribution of each integrin in this context may take considerable efforts. Despite the complexity, these findings elucidate why ECM implantation can enhance muscle regeneration³⁷.

The presently disclosed technology is broadly applicable to muscle diseases involving SC niche dysfunction. Given the role of integrin in other stem cell populations^{19,39}, the presently disclosed technology is expected to have broader implications for aging and decline of function of stem cells in general.

This presently disclosed technology is illustrated by the following examples which should not be considered as limiting.

Materials and Methods

Animal Studies.

Animal experiments in this study were performed in accordance with protocols approved by the Institutional Animal Care and Use Committee (IACUC) of the Carnegie Institution for Science (Permit number A3861-01). The Pax7^{cre-ERT2(CE)} allele (B6; 129-Pax7^{tm2.1(cre/ERT2)}Fan/J) has been described¹⁸. The Itgb1^{f} allele (B6; 129-Itgb1tm1Efu/)¹⁹ and the R26R^{lacZ} (B6.129S4-Gt(ROSA)26Sortm1Sor/J)⁴⁰ and R26R^{YFP} (B6.129X1-Gt(ROSA)26Sor^{tm1(EYFP)}Cos/J)⁴¹ reporter mice were obtained from the Jackson Laboratory. The experimental mice used in this study were Pax7^{CE/+}; Itgb1^{f/f}; R26R^{LacZ/LacZ}, Pax7^{CE/+}; Itgb1^{f/f}; R26R^{YFP/YFP}, or Pax7^{CE/+}; Itgb1^{f/f}, referred to as Itgb1^{-/-}. Reporter alleles were chosen based on the assay: YFP (yellow fluorescent protein) is preferable for immunofluorescence, and necessary for live-imaging and FACS sorting, while LacZ is useful for histological analyses. Controls used were Pax7^{CE/+}; R26R^{LacZ/LacZ}, Pax7^{CE/+}; R26R^{YFP/YFP}, or Pax7^{CE}. For young versus aged comparisons, mice were used at 3-6 month of age (young) or 18-24 month of age (aged). For non-lineage marked SC studies, aged C56/BL6 mice were used (JAX and NIH). Sex was mixed. For dystrophic muscle studies, control and mdx male mice of C57BL/10 background (JAX) were used at 3-4 months of age.

Mice were given tamoxifen (tmx, 20 mg/ml in corn oil (Sigma)) at 3 mg per 40 g body weight per intraperitoneal injection, once a day consecutively for 5 days. All experiments except where noted were conducted 3 d after the final injection. For injury, mice were anesthetized using 2,2,2-Tribromoethanol (Sigma), which was dissolved in 2-methyl-2-butanol (Sigma) as 100% (w/v) stock solution, diluted 1:80 in PBS, and injected intraperitoneally at 10 μl per g body weight. For Itgb1^{-/-} vs. control injury, 50 μl of 10 μM cardiotoxin (CTX; Sigma) was injected using an insulin syringe (U-100; Becton Dickinson) into TA (Tibialis Anterior) muscles. Animals were then harvested at post injury time points stated herein. For short-term daily in vivo proliferation assay, EdU (ehtynyl deoxyuridine, Invitrogen) was given by intraperitoneal injection at 0.1 mg per 20 g bodyweight per injection, at 2, 3, 4, or 5 d after injury. Animals were harvested 24 h after injection. For long-term BrdU incorporation, mice were fed with 0.8 mg/ml of BrdU in drinking water for 1 month. For labeling Pax7⁺ cells, both aged and young Pax7^{CE}; R26R^{YFP/YFP} animals were injected with tmx as described above and harvested for analysis or single fiber culture. For needle tract injury, TA muscles were injured as described previously⁶. Two d post injury, TA muscles received 6×10 μl anti-TLR2 [TS2/16] (10 μg/ml), 60 μl vehicle control (mouse IgG, 10 μg/ml), RGD peptide (10 μg/ml), or scrambled peptide (10 μg/ml) by intramuscular injections. Muscles were harvested 3 d after these injections for analysis. For mdx mice, 10 μg (in 25 ul) of vehicle control IgG or TS2/16 were injected to each TA muscles, either by single injection or repeated 4 times weekly.

Antibodies and Recombinant Proteins.

Antibodies against pan-β1-integrin, Cleaved Caspase-3, Parvin, pp38, pAkt, Akt, pErk1/2, Erk1/2, pFAK, FAK, pFGFR, and FGFR1 were from Cell Signaling (4706, 5A1E, 4026, 9216, 4060, 4691, 4370, 4695, 8556, 13009, 3471, and 9740, respectively). Anti-GAPDH antibody was from Chemicon (MAB374). Antibodies against activated β1-integrin and Paxillin were from BD Biosciences (9EG7 and 610619, respectively). For detecting β1-integrin by Western blot and Par3 in SCs on single fibers, MAB1997 and 07-330, respectively, from Millipore were used. Anti-TLR2 [TS2/16] and control mouse IgG were from Abcam (ab1119333 and ab37355, respectively). Anti-BrdU antibody was from Exalpha (A250P). GFP antibodies were from Invitrogen (G10362, rabbit) and Aves (GFP-1020, chick). Antibodies against eMyHC, MHC, and Pax7 were from Developmental Studies Hybridoma Bank (F1.652, MF20, and PAX7, respectively). Anti-PAX7 of rabbit origin (PA1-117; Thermo) was also used for co-staining with Abs of mouse origin. Anti-Laminin and anti-Vinculin were from Sigma (L9393 and V9131, respectively). Antibodies against Cyclin A, Cyclin BI, Cyclin D1, Cyclin D2, Cyclin D3, Cyclin E, ILK, and MyoD were from Santa Cruz Biotech (sc-596, sc-245, sc-717, sc-593, sc-182, sc-198, sc-20019, sc-81471, and sc-304, respectively). RGD and scrambled peptides were from Santa Cruz Biotech and AnaSpec. Carrier-free FGF-2 was purchased from R&D systems and dissolved and stored at −80° C. The working dilution of the above antibodies and concentrations of recombinant proteins were typically used as recommended by the companies, unless otherwise specified below or in the text and legends in assay-dependent manners.

Muscle Sample Processing.

TA muscles were harvested, fixed for 8 min in ice cold 4% paraformaldehyde (EMS) in phosphate buffered saline (PBS), incubated sequentially in 10% and 20% sucrose/PBS overnight, frozen in isopentane (Sigma)/liquid nitrogen, and stored at −80° C. freezer until cryosectioning. Cross-sections (10 μm) were stained with Haematoxylin and Eosin (H&E, Surgipath), Gomori's one-step trichrome staining kit (Polysciences), or subjected to X-gal (Sigma) reactions as described previously⁴², or used for immunostaining and EdU reactions as described herein.

SC Isolation and Myoblast Culture.

YFP-marked cells were isolated following a published protocol¹⁷. Briefly, for SC preparation, muscles were dissected and incubated in 0.2% Collagenase Type I (Sigma) in DMEM (Gibco) at 37° C. with gentle shaking for 1.5 h. Muscle was then triturated in 10% FBS in DMEM, washed with PBS, and incubated in 0.2% Dispase (Gibco) in DMEM at 37° C. with gentle shaking for 30 minutes. Cells were filtered through a 70 μm cell strainer (VWR) and subjected to cell sorting using the BD FACS ARIA III, gating first for cell size using forward and side scatter, and then for YFP fluorescence. FACS Diva (for cell isolation) software was used. Cells were then used for downstream analyses. For cell cycle analysis, migration, and programmed cell death assays, as well as protein extracts for Western blotting, or RNA isolation for RNA-seq over a time course, cells were cultured in ‘minimal’ growth media (10% Horse Serum (HS), 1% Pen/Strep, 1% Glutamax in DMEM with high glucose; GIBCO) on Matrigel (BD biosciences) coated tissue culture dishes. For differentiation and fusion, cells were cultured in media containing 2% HS on matrigel. For Erk and Akt signaling assays, when considerably more cells were needed, cells were expanded as myoblast cultures in ‘enriched’ growth media (20% Fetal Bovine Serum, 5% Horse Serum, 1% Pen/Strep, 1% Glutamax (Gibco), 0.1% chick embryo extract (MP biomedicals), and 10 ng/ml FGF (R&D systems)) on Matrigel, until sufficient cell numbers were reached, typically in 5-7 days. All cell cultures were placed in 37° C. tissue culture incubators with 5% C02.

Myofibers with associated SCs were isolated from extensor digitorum longus (EDL) muscles by 1.5 h digestion in 0.2% Collagenase Type I in DMEM at 37° C. The digested muscle was then transferred to tissue culture dishes containing DMEM, 1% Pen/Strep, and 1% Glutamax. Live myofibers were isolated with a fire polished glass pipette. Isolation of individual myofibers by pipette was repeated to remove dead myofibers and cellular debris. They were either immediately fixed for assay (e.g. probe for activated β1-integrin) or placed in DMEM, 10% Horse Serum, 1% Penn/Strep, and 1% Glutamax with daily medium and reagent changes. This minimal growth medium contains no additional additives. Depending on the assays, myofibers were cultured for different amount of time before fixation. For Par3, pp38, FGFR1, and pFGFR analysis, myofibers were cultured for 36 h. For self-renewal assays with Pax7 and MyoD expression, 48-96 h cultures were used (specified in text or figure legend). They were either cultured with or without FGF-2 (10 ng/ml), and with or without IgG or TS2/16 (10 μg/ml). Phosphatase Inhibitor Cocktail Set II (Calbiochem) was used as directed during fixation.

For FGF and fibronectin stimulation, the condition described previously for other cell types^{18,21} was modified. The day of experiment, control and Itgb1^{-/-} myoblasts were detached by 2 mM EDTA in serum-free base-media (SFBM, high glucose DMEM, 0.5% BSA, Pen/Strep), washed twice and cultured in SFBM on petri-dish as suspension for 2 h to minimize residual effect of growth factors and contact-dependent signaling from prior culture condition. 50,000 cells were then transferred to each well of a 12-well dish containing SFBM with or without fibronectin (10 μg/ml) for 20 min. The wells were either coated with Sigma-cote (to prevent attachment) or pre-coated with fibronectin (10 μg/ml overnight). Specified concentrations of FGF-2 were then added and cells were harvested 10 min later for Western blots. Blots were first probed with anti-pErk1/2 and anti-pAkt, followed by HRP-conjugated secondary Ab and ECL detection (Amershame). Blots were then stripped and re-probed with anti-Erk1/2 and anti-Akt. Fold of stimulation is presented as pErk/Erk and pAkt/Akt ratios relative to pErk/Ekr and pAkt/Akt ratios, respectively, of control cells in SFBM. The pErk/Ekr and pAkt/Akt ratios of control cells in SFBM are used as the normalization denominator and set at an arbitrary unit of 1-fold.

Immunostaining.

Cells or muscle sections were fixed for 10 min in 4% paraformaldehyde, permeabilized with 0.1% Triton-X-100 (Sigma)/PBS for 15 min at room temperature (RT), rinsed with wash buffer (0.05% Triton-X 100/PBS), treated with blocking buffer (10% Normal Goat Serum (Genetex) and 1% Blocking powder (Perkin Elmer) in wash buffer) for 1-2 hr, prior to incubation with primary antibodies diluted in blocking buffer overnight at 4° C. Primary antibodies against following antigens were diluted as follows: activated 11-integrin, 1:200; β1-integrin, 1:200; BrdU, 1:200; Cleaved Caspase-3, 1:400; eMyHC, 1:200; GFP (used to detect YFP), 1:500 (rabbit) or 1:200 (chick); ILK, 1:50; Laminin, 1:2000; MHC, 1:20; MyoD, 1:50; Parvin, 1:400; Pax7, 1:20; Paxillin, 1:250; pp38, 1:50; Vinculin, 1:400. Cells or muscle sections were washed with wash buffer and incubated with appropriate Alexa-Fluor-conjugated secondary antibodies (1:1,000, Invitrogen) with various fluorescent wavelengths in blocking buffer for 1 h at RT. After wash and incubation with DAPI (1 μg/ml for 5 min), slides were then mounted with Fluoromount-G (SouthernBiotech) and coverslips (VWR). For BrdU detection, slides treated were with antigen unmasking solution (Vector) by boiling for 10 min prior to blocking and primary antibody. For EdU detection, the Click-iT reaction kit (Invitrogen) was used prior to incubation in DAPI.

Western Blot.

For protein extraction, cells were lysed in T-Per Tissue Protein Extraction Reagent (Thermo), 1 mM PMSF (Sigma), lx Halt Phosphatase Inhibitor Cocktail (Thermo), and Complete Protease Inhibitor Tablet (Roche). Total protein extract was resolved by SDS-PAGE on precast gels (Bio-Rad) and then transferred to Immuno-blot PVDF Membranes (Bio-Rad) using a Bio-Rad mini-Protein II Transfer system. Membranes were rinsed in 0.1% Tween (Sigma)/TBS (20 mM Tris-HCl pH 7.5, 150 mM NaCl), blocked for 1 h at RT in 5% low-fat milk (Carnation)/0.1% Tween/TBS (blocking buffer), then incubated with primary antibodies in blocking buffer overnight at 4° C. Primary antibodies against following antigens were used at specified dilutions: β1-integrin, 1:1000 (Millipore); Cyclin A, 1:200; Cyclin B, 1:200; Cyclin D1, 1:200; Cyclin D2, 1:200; Cyclin D3, 1:200; Cyclin E, 1:200; Gapdh, 1:5000; pErk, Erk, pAkt, Akt, FAK, and pFAK, all at 1:1000. After washing in 0.1% Tween/TBS, appropriate secondary antibodies (Amersham, Invitrogen, and Bio-rad) were diluted 1:10,000 in blocking buffer and incubated for 1 h at RT.

Co-Immunoprecipitation.

HEK293T cells (Clontech, tested negative for mycoplasma contamination by MycoProbe Mycoplasma Detection Kit (R&D Systems)) were plated on fibronectin-coated 6-well dishes and transfected with pcDNA3-FGFR1-flag and/or pcDNA3-Itgb1-HA by lipofectamine2000 (Invitrogen) overnight according to manufacturer's manual. Next day, they were incubated in media containing 5 μg/ml of IgG or TS2/16 for 1 h at 4° C. Cells were washed with cold PBS and lysed in 1% NP40 (50 mM HEPES, 150 mM NaCl, 10% glycerol, protease inhibitors (Thermo)) for 30 min at 4° C. Lysates were clarified by centrifugation, and subjected to immunoprecipition by anti-HA affinity matrix (Pierce) or anti-Flag affinity matrix (Sigma) for 2 h at 4° C. Affinity matrix were washed and proteins were eluted in 2×SDS-PAGE sample buffer for Western blotting using anti-Flag and anti-HA antibodies (Sigma).

Force Measurements and Fatigue Analysis.

In situ force measurements of TA muscles were conducted as done previously^{43,44}, and the data were analyzed using the 1300A Whole Animal System (Aurora Scientific). Mice were anaesthetized with isoflurane and placed on isothermal stage. Intact TA muscles were dissected and constantly immersed in Ringer's solution. Single twitch or tetanic contractions were elicited with electrical stimulations applied by two electrodes placed on either side of the muscle. In all experiments, 0.2 ms pulses at 10 V supramaximal voltage were used. Muscle optimal length (Lo) that allows a maximum isometric twitch force (Pt) was determined by a series of twitch contractions with small variations of the muscle tension. To obtain maximum isometric tetanic force (Po), muscles were stimulated for 300 ms at different frequencies from 50 to 200 Hz. A 1 min recovery period was allowed between stimulations. The muscles were then fatigued at 150 Hz with one contraction per second for 180 s. Muscle wet weight and Lo were used to calculate the cross-sectional area (CSA) of the TA muscle for normalization to obtain specific isometric twitch force sPt (kN/mm²) and sPo (kN/mm²).

Microscopy and Image Processing.

Images of hematoxylin and eosin stained muscle sections were captured from a Nikon 800 microscope with 10× or 20× Plan Apo objectives and Canon EOS T3 camera using EOS Utility image acquisition software. Fluorescent images of muscle sections and single myofibers were wither captured using Leica SP5 confocal equipped with 40×/1.25 Plan Apo oil objectives using Leica image acquisition software, or a Zeiss Axioskope equipped with a 40×/0.5 Plan Apo oil objective and Axiocam camera using Zeiss image acquisition software. Identical exposure times were used and images were processed and scored with blinding using ImageJ64. If necessary, brightness and contrast were adjusted for an entire experimental image set. For quantification of polarized cell markers (Par3, m-cadherin, pp38), each set of images was scored by three individuals with blinding using ImageJ64, only those that were agreed upon by all individuals were included. For the rest, Imaris (Bitplane) was used for three-dimensional rendering of fluorescence data for quantification of polarization. A subset was also randomly selected for quantitative analysis via Imaris to confirm scoring. Cell number, fiber diameter, fiber number, and fiber cross sectional area were determined using ImageJ64 or Fiji using images of a micrometer (VWR) taken under the same magnifications as the sample images as references for imaging field sizes.

FACS Analysis of DNA Content.

For FACS analysis of DNA content, cells were collected at staggered time points post activation (24, 48, 72 h). Cells were centrifuged (2500×g, 4° C., 10 min) and washed twice in cold PBS. Cell pellets were resuspended in solution of 70% cold ethanol and fixed overnight at 4° C. After washing twice with cold PBS, the cells were resuspended in 1 mL of a 1:1 PI solution (0.1 mg/mL propidium iodide in 0.6% Triton-X in PBS (Sigma; P4170)): RNase solution (2 mg/mL in milli-Q H₂O (Sigma; R5125)) and stained in the dark for 45 min. Cells were passed through meshed capped falcon tubes prior to running on FACS machine to avoid clumping. FACS analysis was carried out with a BD FACS ARIA III machine and FACS Diva software, gating first for cell size and using forward and side scatter and then gating for YFP+ cells using the 488 channel. ModFit LT V2.2.11 was used to analyze percentages of each phase of the cell cycle, G₁, S, and G₂/M. All data were determined to have “good” RCS, measurement of fit.

RNA-Seq Analysis.

For RNA-seq analysis, FACS isolated SCs were cultured in minimal growth media on Matrigel-coated plates. At 24, 48 and 72 h, RNA was isolated using the Arcturus PicoPure RNA isolation kit (Applied Biosystems). The Ovation RNA-Seq System (NuGEN) was used to prepare amplified cDNA. Libraries for single-end sequencing were prepared using Illumina's TruSeqDNA sample prep kit LT. Sequencing was carried out on an Illumina HiSeq2000 to generate single-end 100 bp reads, which were aligned to the mouse genome (mm9) using TopHat (v2.0.7). Cufflinks (v2.1.1) was used for differential expression analysis. Only genes that displayed “yes” significance as determined by Cuffdiff 2 were analyzed further: if P value of observed change in FPKM was greater than the FDR (False discovery rate) after Benjamini-Hochberg correction for multiple-testing⁴⁵.

Statistical Analyses Quantitative values are expressed as the mean±standard deviation (s.d.) or standard error of the mean (s.e.m.). Statistical differences between groups were determined using an Excel spreadsheet by using a t-test for two-tailed paired comparison. When comparing more than two sets, one-way or two-way ANOVA with Tukey's post hoc test was performed using GraphPad Prism 6. P<0.05 was determined to be significant for all experiments. All experiments requiring the use of animals or animals to derive cells were subject to randomization based on litter. No animals or samples were excluded from the study. Sample size was predetermined based on variability observed in prior experiments and preliminary data. Investigators were not blinded to outcome assessment.

### Example 1

SCs without β1-Integrin Cannot Maintain Quiescence

To define the function of β1-integrin in adult SCs, a Pax7^{Cre-ERT2(CE)} driver¹⁸

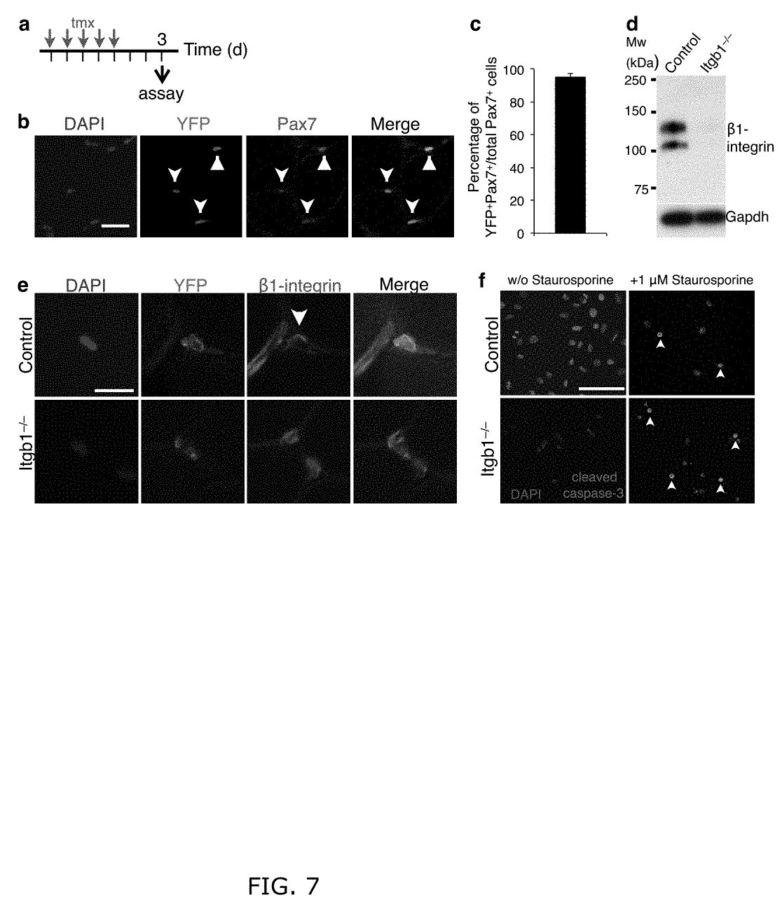

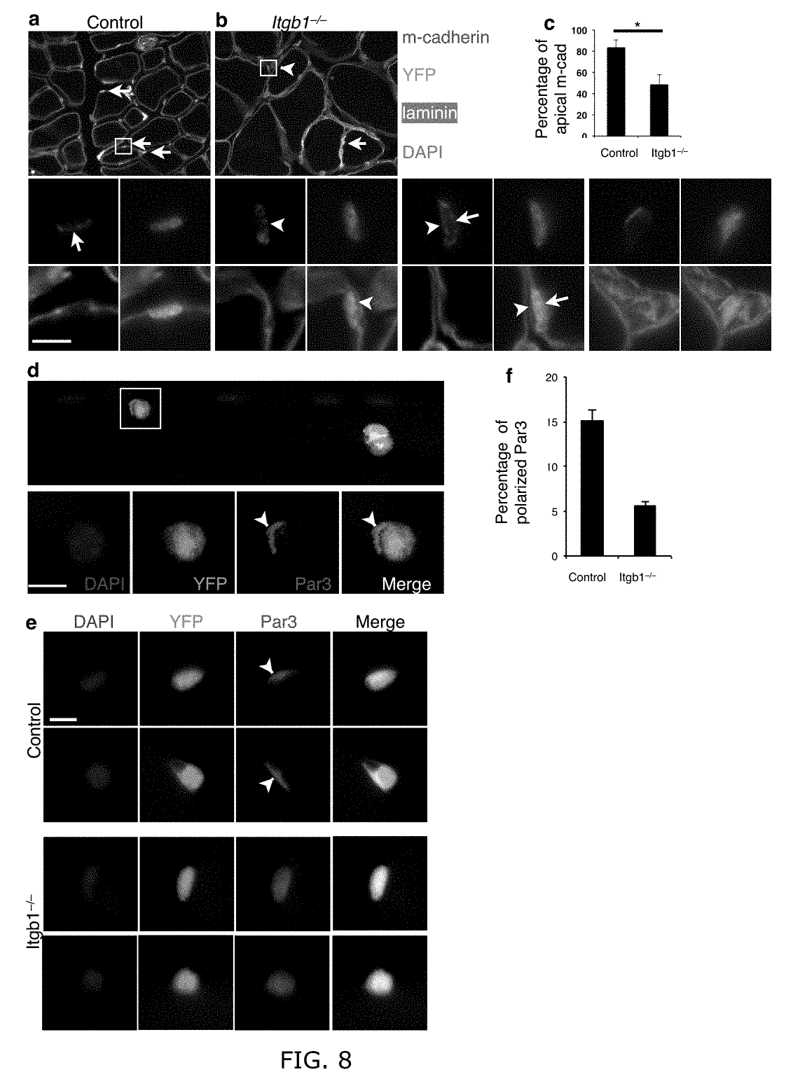

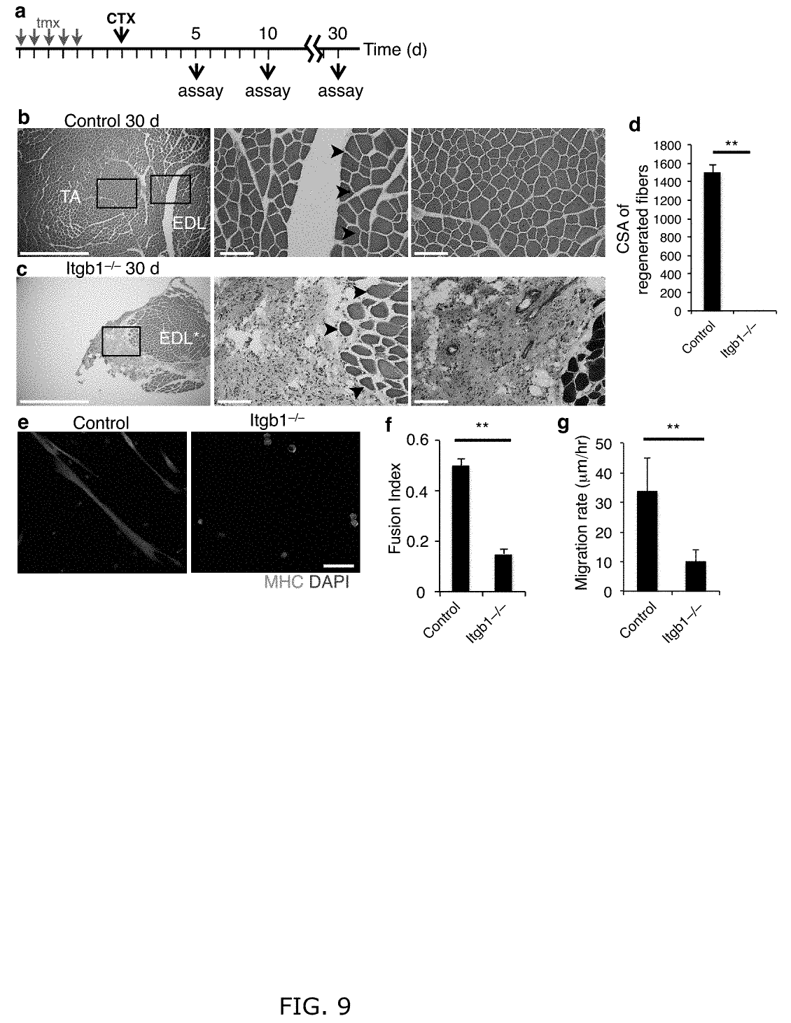

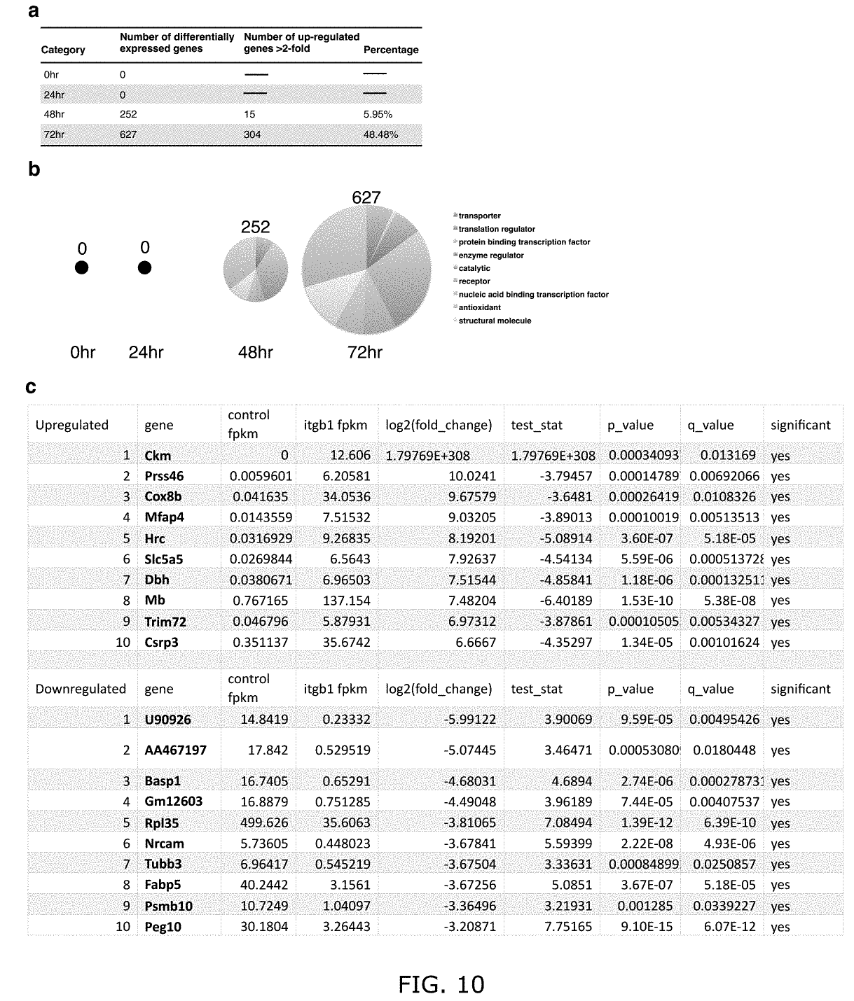

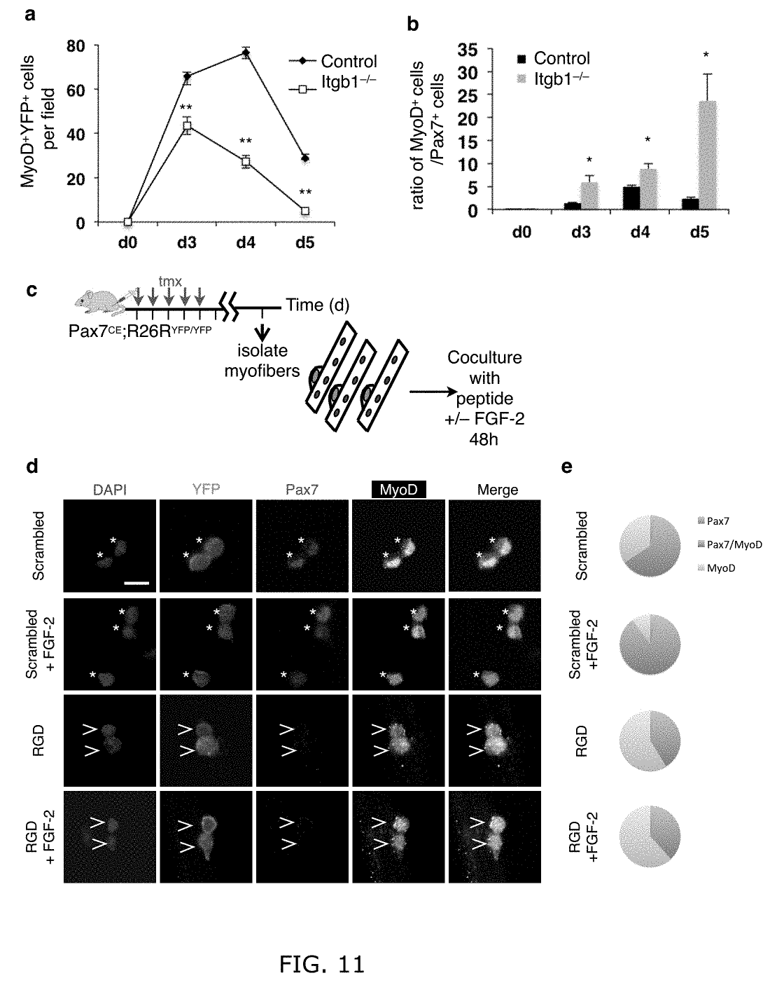

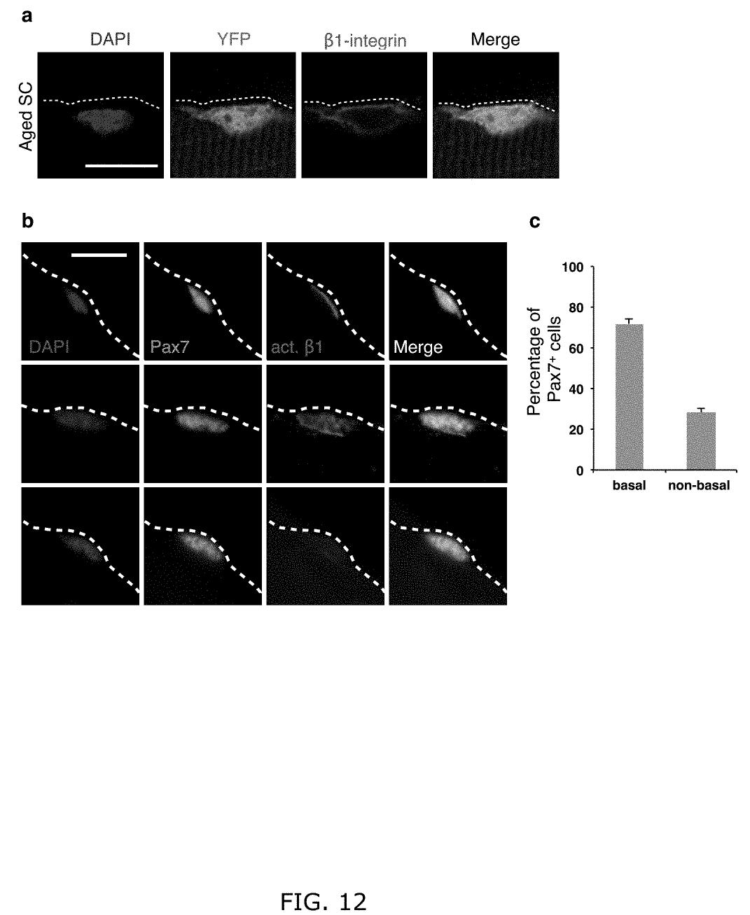

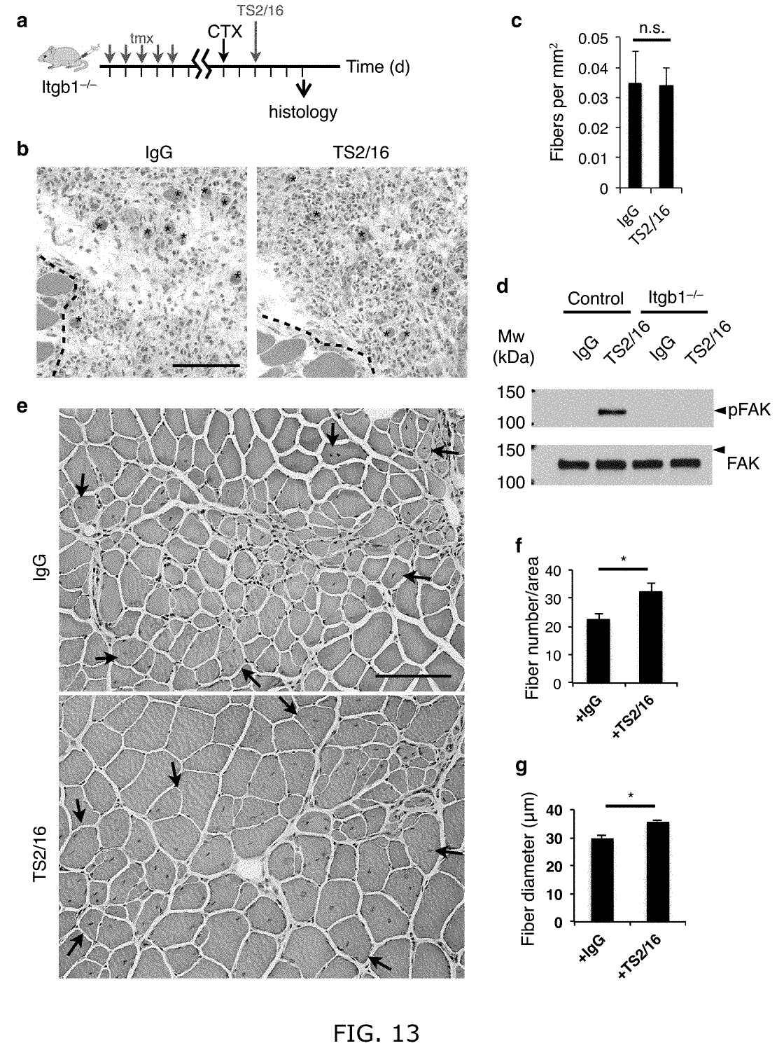

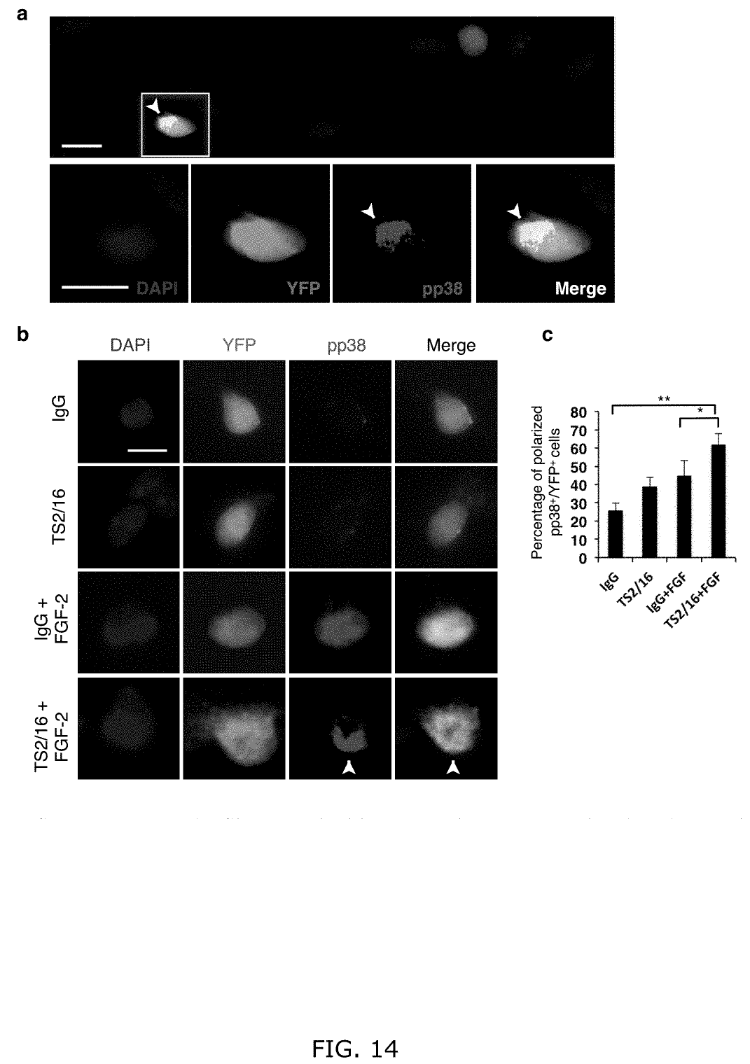

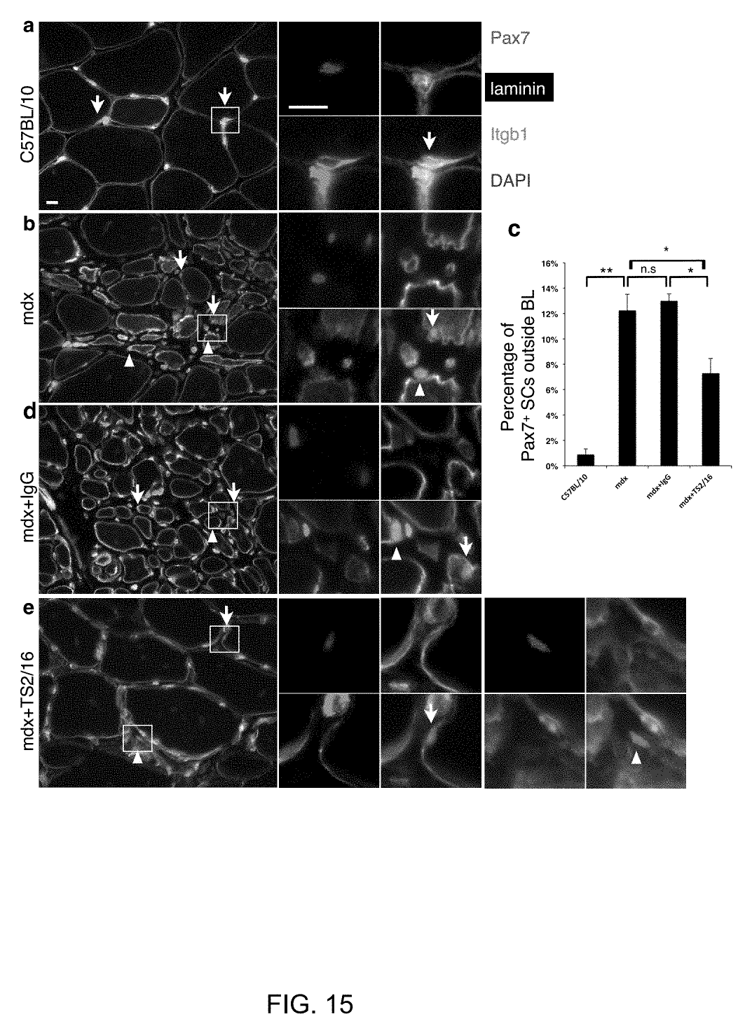

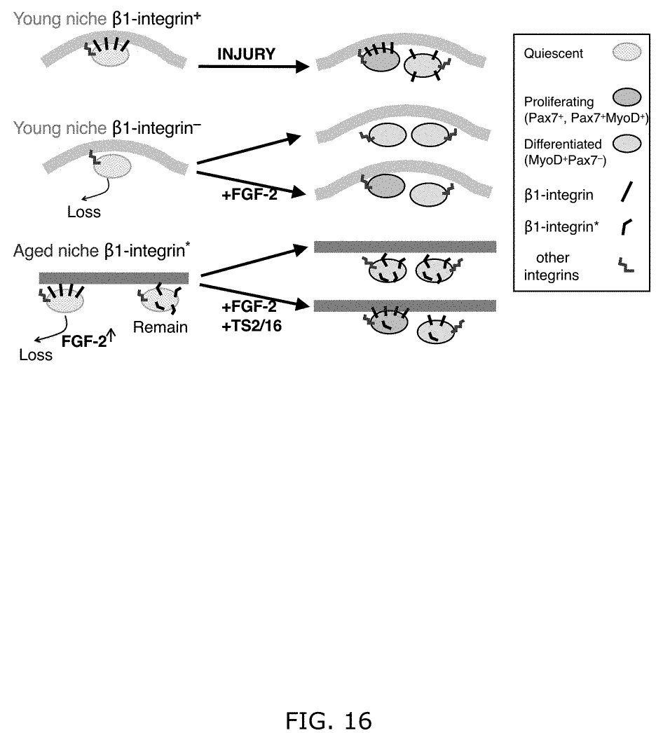

for tamoxifen (tmx) inducible gene inactivation of a Itgb1 conditional allele (Itgb1^{f};⁹) was used in SCs. Cre reporter alleles R26R^{YFP} or R26R^{LacZ} were included for lineage marking and assessing recombination efficiency (FIG. 7a-c;¹⁸). Three days (3 d) after tmx regimen, YFP⁺ conditional mutant (Itgb1^{-/-}) SCs no longer had detectable β1-integrin (FIG. 7d,e) but maintained Pax7 expression, compared to control SCs (FIG. 1a). To assess whether β1-integrin loss affected SC niche occupancy, lineage-marked cells were followed up to 180 d (FIG. 1b-d). The number of Itgb1^{-/-} SCs was unchanged compared to that of controls 7 d after tmx regimen, but reduced to about half of that in controls at 21 d. Itgb1^{-/-} SCs continued to decline over time, but were not completely lost by 180 d. Programmed cell death (PCD) of Itgb1^{-/-} SCs was not detected at these time points (FIG. 7f). Instead, lineage-labeled myofibers suggest mutant SC loss via differentiation and incorporation into existing muscle fibers (FIG. 1b,c).

To test whether their slow loss was indeed due to spurious activation and differentiation, continuous BrdU administration was performed for 30 d. A significant portion of Itgb1^{-/-} SCs incorporated BrdU during this period, revealing aberrant cell cycle entry (FIG. 1e). A greater fraction of MyoD⁺ cells was also detected in the mutant, compared to the control (FIG. 1f), supporting spurious differentiation. Given that there was no increase in SCs, those that became activated likely had limited proliferative capacity. Furthermore, polarity proteins m-cadherin and Par3 were not localized appropriately in Itgb1^{-/-} SCs (FIG. 8), suggesting mutant SC loss is associated with polarity defects. It was concluded that β1-integrin senses quiescent SC-niche to maintain polarity and prevent spurious activation and differentiation.

### Example 2

SCs without β1-Integrin Cannot Sustain Proliferation Upon Injury

To assess whether Itgb1^{-/-} SCs could support regeneration, muscle injury was conducted by cardiotoxin at 3 d post tmx (FIG. 9a). These and all subsequent assays were performed when mutant SC number was normal in order to appropriately analyze the separate role of β1-integrin in SC-driven muscle regeneration. Severely defective regeneration was observed at 5, 10, and 30 d post-injury (FIGS. 1g-i and 9b-d). Consistent with embryonic studies¹⁶, adult Itgb1^{-/-} myoblasts migrated and fused poorly in vitro (FIG. 9e-g), which explains the reduced diameter of regenerated fibers (FIG. 1i).

The drastic reduction in regenerated fiber numbers (FIG. 1h) in the mutant suggests a SC expansion defect. To test this, proliferative cells were pulsed with EdU from 2 to 5 d daily after injury. Itgb1^{-/-} SCs proliferated normally at d 2, but did not sustain the same proliferative rates in ensuing days as control SCs (FIG. 2a,b). Although PCD did not appear to be a contributor to reduced mutant cells (FIG. 7f), other cell elimination processes cannot be excluded. To ascertain the molecular changes in Itgb1^{-/-} SCs, cell cycle analyses of FACS-isolated YFP⁺ control and mutant SCs²⁰ in culture was performed (FIG. 2c-f). Compared to control cells, mutant cells had lower levels of cyclins D1 and D2, and misregulated cell cycle showing an increased fraction of G1 and a decreased faction in S phase at 72 h. Gene expression profiles of control and mutant SCs showed no differences up to 24 h in culture, but became progressively different at 48 and 72 h (FIG. 10). At 72 h, many genes associated with muscle differentiation were upregulated in mutant cells (FIG. 2g). These data are consistent with the role of β1-integrin in sustaining cyclin D levels and G1/S transition in other cell types^{13,21}

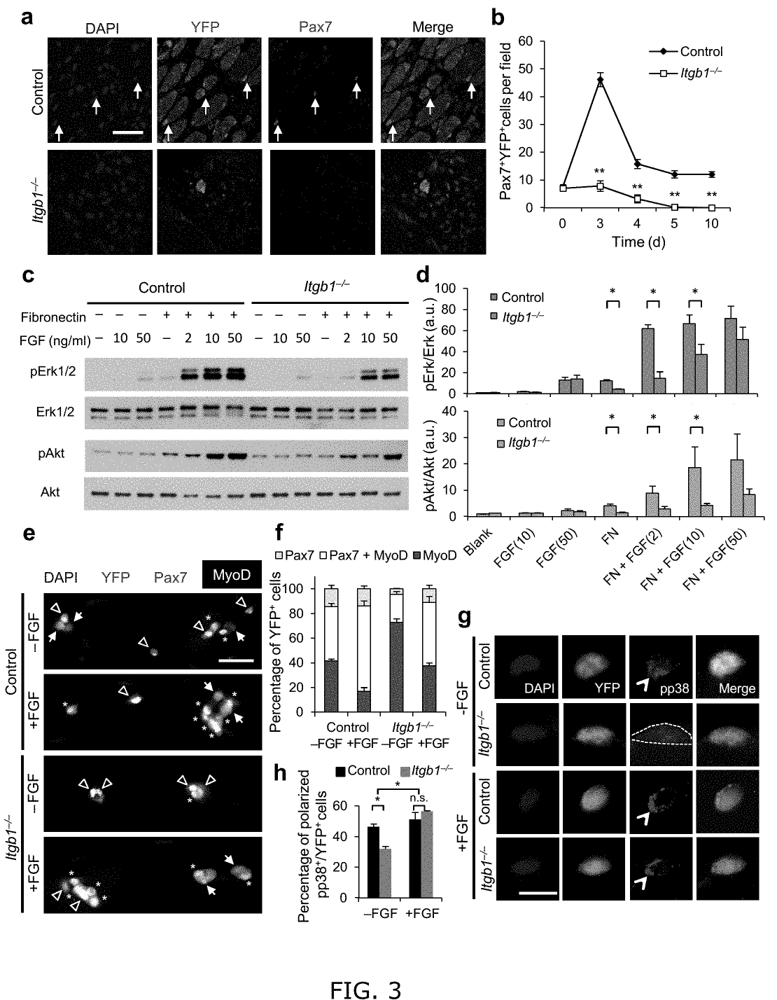

To determine whether there was a defect in SC self-renewal, Pax7⁺ cells in the regenerative area of Itgb1^{-/-} mice were monitored. Pax7⁺ cell numbers were significantly reduced at d 3 and d 4, and not present after d 5 (FIG. 3a,b). Although there were fewer MyoD⁺ mutant cells, ratios of MyoD⁺ to Pax7⁺ mutant cells were higher than those of control cells (FIG. 11a,b), indicating that Itgb1^{-/-} SCs turned off Pax7 and failed to self-renew. Thus, β1-integrin is required for SC proliferation and self-renewal to drive muscle regeneration.

### Example 3

β1-Integrin Crosstalks with FGF Signaling in SCs

Activation of integrins by the ECM cooperates with receptor tyrosine kinases (RTKs), including FGFR, to activate Erk MAP kinases and drive proliferation in many cell types^{12,13,21-24}. FGF-2 stimulates myoblast proliferation via the same effectors²⁵, which may involve integrin signaling. Itgb1^{-/-} myoblasts were examined for defects in FGF-2 responsiveness with or without fibronectin, an ECM molecule originally used to demonstrate integrin-RTK crosstalk^{21,24}. The phosphorylation levels of their common downstream effectors, Erk and Akt (pErk1, pErk2 and pAkt) were assessed (FIG. 3c,d). Control cells responded better to FGF-2 in the presence of fibronectin; low levels of FGF-2 was sufficient to phosphorylate Erk and, to a lesser extent Akt. In contrast, fibronectin or high levels of FGF-2 alone only weakly activated Erk and Akt. Mutant cells were indeed defective in responding to FGF-2 in the presence of fibronectin. Importantly, high levels of FGF-2 could partially restore cooperativity, likely due to compensation by other fibronectin-binding integrins in Itgb1^{-/-} cells.

Next, the relevance of the cooperativity between integrin and FGF signaling in the SC was assessed. Using the single myofiber assay⁹ to examine SC fates, it was determined that the majority of control SCs maintained Pax7 expression (Pax7⁺MyoD⁻ and Pax7⁺MyoD⁺). Addition of FGF increased the Pax7⁺MyoD⁺ expanding population but did not alter the Pax7⁺MyoD⁻ self-renewed fraction (FIG. 3e,f). In contrast, the majority of mutant SCs were committed to differentiate, expressing only MyoD, at the expense of Pax7⁺MyoD⁺ and Pax7⁺MyoD⁻ fractions. The latter two populations were partially rescued by FGF-2 addition, again suggesting compensation by other integrins. As SCs express almost all integrin subtypes²⁶, the RGD peptide, a broad-spectrum integrin-binding competitor that interferes with ECM engagement¹⁴, was tested for whether it was sufficient to disrupt FGF response. SCs treated with RGD peptide had a reduced fraction of Pax7⁺MyoD⁺ cells, which could not be rescued by FGF-2 (FIG. 11c-e). Thus, RGD-binding integrins in the SC, including those containing β1-integrin, cooperate with FGF-2. SC self-renewal has been associated with FGF stimulation of asymmetric p38α/β MAPK phosphorylation^{9,27}. Fewer Itgb1^{-/-} SCs displayed polarized pp38, relative to controls, and FGF-2 could increase the fraction of mutant SCs with polarized pp38 (FIG. 3g,h). While wishing to be bound to any specific mechanism of action, the results of the present disclosure suggest a mechanism for β1-integrin in sustaining SC expansion and self-renewal.

### Example 4

Aged SCs are Defective in Integrin Activity

The characteristics of Itgb1^{-/-} SCs are similar to aged SCs; both are gradually lost from the niche², cannot sustain proliferation²⁸, are committed to differentiation⁵, and are defective in self-renewal⁹. Given that ECM composition²⁹ and stiffness³⁰ change in aged muscles, it was not clear whether the aged environment impacts β1-integrin or overall integrin activity, thereby desensitizing aged SCs to FGF-2⁹. To probe for changes in integrin activity in aged SCs, an antibody (9EG7) that recognizes the “high-affinity” ligand-bound active β1-integrin^{31,32} on young and aged SCs was used. The majority of young myofiber-associated SCs displayed well-aligned basal membrane-bound β1-integrin in its active conformation, mirroring total β1-integrin (compare FIG. 4a to FIG. 1a). Despite a basally localized pattern of total β1-integrin (FIG. 12a), more aged SCs displayed active β1-integrin in abnormal patterns: in disorganized puncta or undetectable (FIG. 4a,b). To ascertain age-associated changes in overall integrin activity, spatial patterns of their common effectors were monitored: integrin-linked kinase (ILK), parvin, and paxillin (FIG. 4c). ILK, parvin and paxillin were localized to the laminar side in young SCs as expected. In contrast, aged SCs showed disorganized distribution patterns of these effectors. Vinculin, which binds to actin but not directly to integrin³³, encircled young SCs and this pattern was slightly disorganized in aged SCs. Thus, aged SCs display abnormal localization and disparate changes of common integrin effectors, reflecting a dysregulation of overall integrin activity.

If dysregulated integrin signaling underlies the dysfunction of aged SCs, activating β1-integrin alone may be sufficient for rescue. To test this, injured muscles were injected with a β1-integrin-activating antibody TS2/16 (Ref. 34) (FIG. 4d-f). Robust muscle regeneration was observed in control young mice injected with vehicle (YV). The RGD peptide repressed muscle regeneration in young mice (YI). While TS2/16 did not enhance the already robust regeneration in young animals (YA), it improved regeneration in aged mice (AA) to a level comparable to the young. As controls, TS2/16 did not rescue regeneration of Itgb1^{-/-} muscle, but could activate β1-integrin-dependent signaling (FIG. 13a-d). Thirty days later, regenerated muscles still had measurable improvement by a single dose of TS2/16 (FIG. 13e-g).

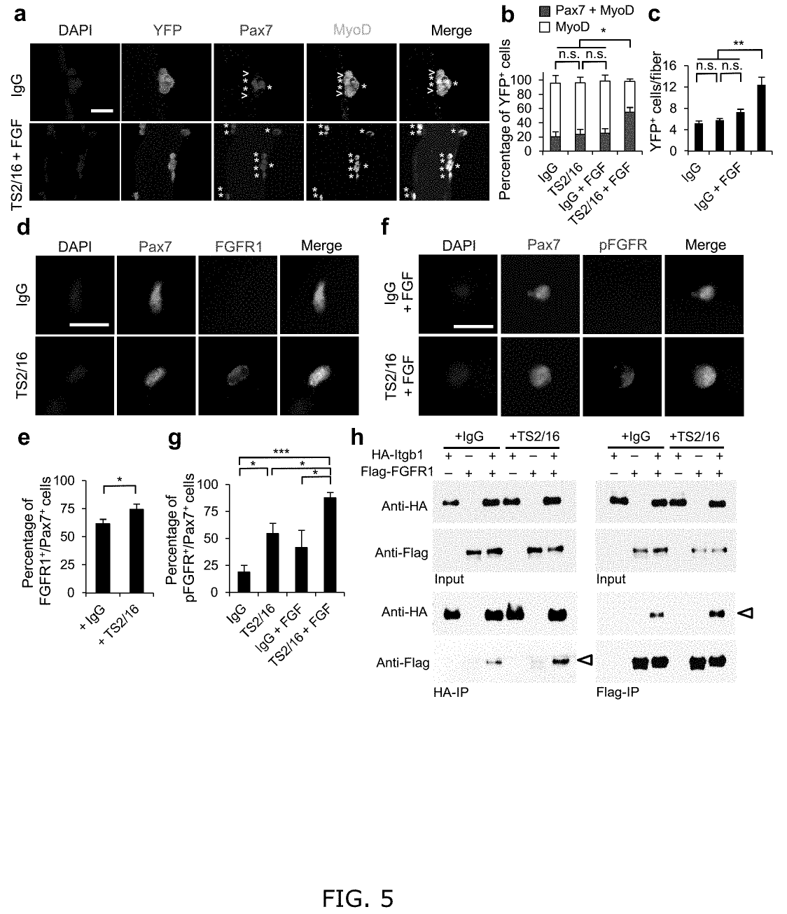

To determine whether TS2/16 improves SC function, it was applied to myofiber-associated aged SCs and their expansion and Pax7 expression was assessed. Neither the IgG control, nor TS2/16 or FGF-2 treatment alone showed an effect. TS2/16 and FGF-2 together increased the fraction of Pax7⁺ cells, the number of fiber-associated myogenic cells, and the fraction of SCs displaying polarized pp38 (FIG. 5a-c and FIG. 14). Since TS2/16 alone is sufficient in vivo, it was surmised that muscle damage releases a sufficient amount of FGF-2 to cooperate with TS2/16-activated β1-integrin in aged SCs. Mechanistically, it was found that TS2/16 increased the fraction of aged SCs on myofibers with detectable FGFR1, relative to control IgG (FIG. 5d,e). Although not sufficient to enhance expansion, TS2/16 alone increased the proportion of aged SCs with detectable phosphorylated FGFR (pFGFR). TS2/16 and FGF-2 together stimulated pFGFR in almost all aged SCs (FIG. 5f,g), consistent with their dual requirement for enhancing aged SC expansion. Associations between FGFR and α-integrin³⁵ or αvβ3-integrin³⁶ were suggested to underlie FGF-integrin cooperativity. It is shown here that FGFR1 can associate with β1-integrin, and that TS2/16 enhances their association (FIG. 5h). These data indicate that TS2/16 operates at multiple layers to enhance FGF signaling and restore the responsiveness in aged SCs.

### Example 5

Activating β1-Integrin Improves Dystrophic Muscles

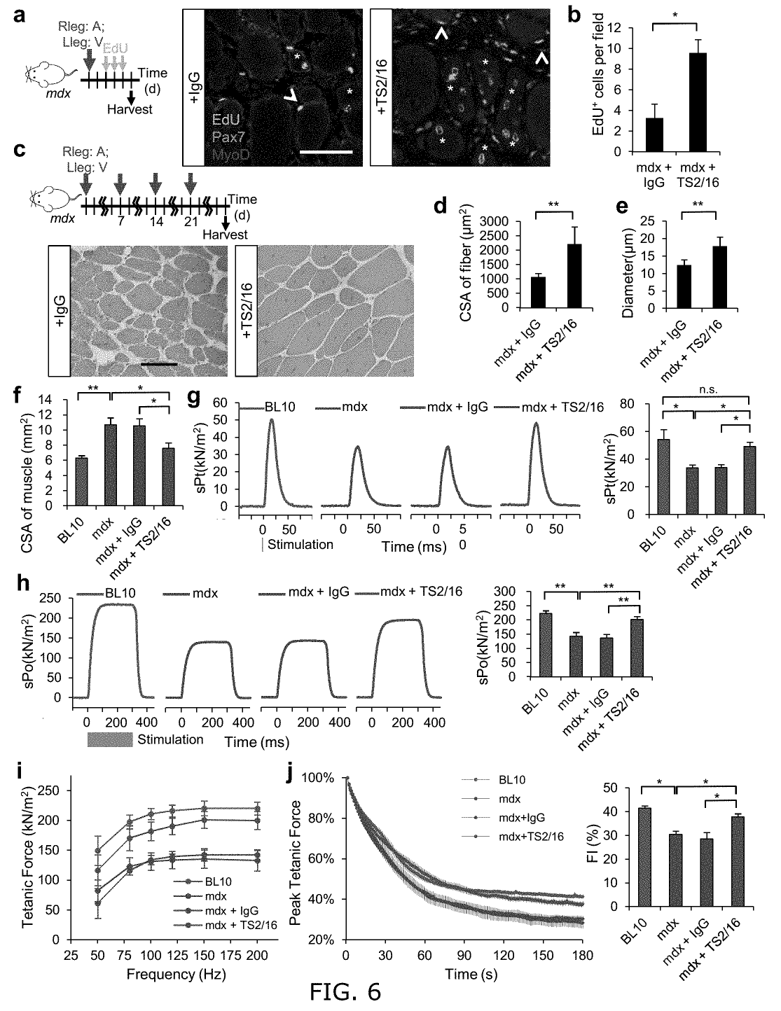

The positive effect of TS2/16 on regeneration in aged environment predicted beneficial use in another context of impaired muscle regeneration: muscular dystrophy. For this, the mdx mouse model was employed, which lacks dystrophin expression due to a nonsense mutation¹⁷. As the mdx muscle contains disorganized ECM, more Pax7⁺ SCs residing outside of the myofiber lamina, relative to those in the control (C57BL/10; FIG. 15a-c) was anticipated. Additionally, mdx SCs associated with myofibers displayed abnormal patterns of active β1-integrin (FIG. 12b,c). TS2/16 was administered into the TA muscle of mdx mice. A single dose was sufficient to promote the expansion of myogenic cells, as shown by increased EdU incorporation 3 d after treatment (FIG. 6a,b). TS2/16 treatment was extended to 4 weekly injections (FIG. 6c) and cross-sectional area and muscle fiber diameter were found to be increased, relative to those treated with control IgG (FIG. 6c-e). The percentage of Pax7+ cells outside of myofiber lamina also was reduced, reflecting an improvement in SC-niche interaction (FIG. 15c-e). As α7β1-integrin binds to laminin, the ECM component that engages with the dystrophin complex, activating β1-integrin likely also enhances muscle fiber integrity in mdx mice via improved connection to the ECM.

To determine whether the above long-term regimen resulted in functional rescue, the contractile properties of IgG- and TS2/16-treated mdx TA muscles in situ were compared; wt and untreated mdx TA muscles were done in parallel for references (FIG. 6f-j). TS2/16-treated mdx muscles had reduced cross sectional areas than those of mdx and IgG-treated mdx muscles, consistent with a reversal from hypertrophic pathology. Significantly, TS2/16-treated muscles showed strength improvements in a variety of measurements, including single twitch force, maximum isometric tetanic force, force-frequency relationship, time to fatigue, and fatigue index. Activating β1-integrin is therefore be a viable therapeutic means to improve muscle repair and function in diseased conditions.

### REFERENCES

1. Ryall, J. G., Schertzer, J. D. & Lynch, G. S. Cellular and molecular mechanisms underlying age-related skeletal muscle wasting and weakness. Biogerontology 9, 213-228 (2008).
2. Chakkalakal, J. V., Jones, K. M., Basson, M. A. & Brack, A. S. The aged niche disrupts muscle stem cell quiescence. Nature 490, 355-360 (2012).
3. Fry, C. S. et al. Inducible depletion of satellite cells in adult, sendentary mice impairs muscle regenerative capacity without affecting sarcopenia. Nature Medicine 21, 76-80 (2015).
4. Brack, A. S. et al. Increased Wnt signaling during aging alters muscle stem cell fate and increases fibrosis. Science 317, 807-810 (2007).
5. Cosgrove, B. D. et al. Rejuvenation of the muscle stem cell population restores strength to injured aged muscles. Nature Medicine 20, 255-264 (2014).
6. Conboy, I. M., Conboy, M. J., Smythe, G. M. & Rando, T. A. Notch-mediated restoration of regenerative potential to aged muscle. Science 302, 1575-1577 (2003).
7. Shea, K. L. et al. Sproutyl regulates reversible quiescence of a self-renewing adult muscle stem cell pool during regeneration. Cell Stem Cell 6, 117-129 (2010).
8. Carlson, M. E., Hsu, M. & Conboy, I. M. Imbalance between pSmad3 and Notch induces CDK inhibitors in old muscle stem cells. Nature 454, 528-532 (2008).
9. Bernet, J. D. et al. p38 MAPK signaling underlies a cell-autonomous loss of stem cell self-renewal in skeletal muscle of aged mice. Nature Medicine 20, 265-271 (2014).
10. Price, F. D. et al. Inhibition of JAK-STAT signaling stimulates adult satellite cell function. Nature Medicine 20, 1174-1181 (2014).
11. Tierney, M. T. et al. STAT3 signaling controls satellite cell expansion and skeletal muscle repair. Nature Medicine 20, 1182-1186 (2014).
12. Mori, S. & Takada, Y. Crosstalk between Fibroblast Growth Factor (FGF) Receptor and Integrin through Direct Integrin Binding to FGF and Resulting Integrin-FGF-FGFR Ternary Complex Formation. Medical Sciences 1, 20-36 (2013).
13. Assoian, R. K. & Schwartz, M. A. Coordinate signaling by integrins and receptor tyrosine kinases in the regulation of G1 phase cell-cycle progression. Curr. Opin. Genet. Dev. 11, 48-53 (2001).
14. Hynes, R. O. Integrins: bidirectional, allosteric signaling machines. Cell 110, 673-687 (2002).
15. Helbling-Leclerc, A. et al. Mutations in the laminin alpha 2-chain gene (LAMA2) cause merosin-deficient congenital muscular dystrophy. Nature Genetics 11, 216-218 (1995).
16. Schwander, M. et al. Beta1 integrins regulate myoblast fusion and sarcomere assembly. Developmental Cell 4, 673-685 (2003).
17. Bulfield, G., Siller, W. G., Wight, P. A. & Moore, K. J. X chromosome-linked muscular dystrophy (mdx) in the mouse. Proceedings of the National Academy of Sciences of the United States of America 81, 1189-1192 (1984).
18. Lepper, C., Conway, S. J. & Fan, C.-M. Adult satellite cells and embryonic muscle progenitors have distinct genetic requirements. Nature 460, 627-631 (2009).
19. Raghavan, S., Bauer, C., Mundschau, G., Li, Q. & Fuchs, E. Conditional ablation of beta1 integrin in skin. Severe defects in epidermal proliferation, basement membrane formation, and hair follicle invagination. J Biophys Biochem Cytol 150, 1149-1160 (2000).
20. Webster, M. T. & Fan, C.-M. c-MET regulates myoblast motility and myocyte fusion during adult skeletal muscle regeneration. PLoS ONE 8, e81757 (2013).
21. Roovers, K., Davey, G., Zhu, X., Bottazzi, M. E. & Assoian, R. K. Alpha5beta1 integrin controls cyclin D1 expression by sustaining mitogen-activated protein kinase activity in growth factor-treated cells. Mol. Biol. Cell 10, 3197-3204 (1999).
22. Polanska, U. M., Fernig, D. G. & Kinnunen, T. Extracellular interactome of the FGF receptor-ligand system: Complexities and the relative simplicity of the worm. Developmental Dynamics 238, 277-293 (2009).
23. Murakami, M., Elfenbein, A. & Simons, M. Non-canonical fibroblast growth factor signalling in angiogenesis. Cardiovascular Research 78, 223-231 (2008).
24. Miyamoto, S., Teramoto, H., Gutkind, J. S. & Yamada, K. M. Integrins can collaborate with growth factors for phosphorylation of receptor tyrosine kinases and MAP kinase activation: roles of integrin aggregation and occupancy of receptors. J Biophys Biochem Cytol 135, 1633-1642 (1996).
25. Jones, N. C., Fedorov, Y. V., Rosenthal, R. S. & Olwin, B. B. ERK1/2 is required for myoblast proliferation but is dispensable for muscle gene expression and cell fusion. J. Cell. Physiol. 186, 104-115 (2001).
26. Siegel, A. L., Atchison, K., Fisher, K. E., Davis, G. E. & Cornelison, D. 3D Timelapse Analysis of Muscle Satellite Cell Motility. Stem Cells 27, 2527-2538 (2009).
27. Troy, A. et al. Coordination of satellite cell activation and self-renewal by Par-complex-dependent asymmetric activation of p38α/β MAPK. Cell Stem Cell 11, 541-553 (2012).
28. Conboy, I. M. et al. Rejuvenation of aged progenitor cells by exposure to a young systemic environment. Nature 433, 760-764 (2005).
29. Kragstrup, T. W., Kjaer, M. & Mackey, A. L. Structural, biochemical, cellular, and functional changes in skeletal muscle extracellular matrix with aging. Scandinavian Journal of Medicine & Science in Sports 21, 749-757 (2011).
30. Wood, L. K. et al. Intrinsic stiffness of extracellular matrix increases with age in skeletal muscles of mice. J. Appl. Physiol. 117, 363-369 (2014).
31. Bazzoni, G., Shih, D.-T., Buck, C. A. & Hemler, M. E. Monoclonal Antibody 9EG7 Defines a Novel β1 Integrin Epitope Induced by Soluble Ligand and Manganese, but Inhibited by Calcium. J. Biol. Chem. 270, 25570-25577 (1995).
32. Takagi, J. & Springer, T. A. Integrin activation and structural rearrangement. Immunological Reviews 186, 141-163 (2002).
33. Calderwood, D. A., Shattil, S. J. & Ginsberg, M. H. Integrins and actin filaments: reciprocal regulation of cell adhesion and signaling. J. Biol. Chem. 275, 22607-22610 (2000).
34. Hintermann, E., Bilban, M., Sharabi, A. & Quaranta, V. Inhibitory role of alpha 6 beta 4-associated erbB-2 and phosphoinositide 3-kinase in keratinocyte haptotactic migration dependent on alpha 3 beta 1 integrin. J Biophys Biochem Cytol 153, 465-478 (2001).
35. Toledo, M. S., Suzuki, E., Handa, K. & Hakomori, S. Effect of ganglioside and tetraspanins in microdomains on interaction of integrins with fibroblast growth factor receptor. J. Biol. Chem. 280, 16227-16234 (2005).
36. Sahni, A. & Francis, C. W. Stimulation of endothelial cell proliferation by FGF-2 in the presence of fibrinogen requires alphavbeta3. Blood 104, 3635-3641 (2004).
37. Sicari, B. M. et al. An acellular biologic scaffold promotes skeletal muscle formation in mice and humans with volumetric muscle loss. Sci Transl Med 6, 234ra58 (2014).
38. Fukada, S.-I. et al. Molecular signature of quiescent satellite cells in adult skeletal muscle. Stem Cells 25, 2448-2459 (2007).
39. Tanentzapf, G., Devenport, D., Godt, D. & Brown, N. H. Integrin-dependent anchoring of a stem-cell niche. Nat. Cell Biol. 9, 1413-1418 (2007).
40. Soriano, P. Generalized lacZ expression with the ROSA26 Cre reporter strain. Nature Genetics 21, 70-71 (1999).
41. Srinivas, S. et al. Cre reporter strains produced by targeted insertion of EYFP and ECFP into the ROSA26 locus. BMC Dev. Biol. 1, 4 (2001).
42. Hogan, B., Beddington, R., Cstantini, F. & Lacey, E. Manipulating the mouse embryo. (Cold Spring Harbor Laboratory Press, 1994).
43. Hakim, C. H., Wasala, N. B., Duan, D. Evaluation of Muscle Function of the Extensor Digitorum Longus Muscle Ex vivo and Tibialis Anterior Muscle In situ in Mice. J. Vis. Exp. 21 (2013).
44. Barton, E. R., Khurana, T. S., Lynch, G. S. Measuring isometric force of isolated mouse muscles in vitro. TREAT-NMD. (2008).
45. Trapnell. C. et al. Differential analysis of gene regulation at transcript resolution with RNA-seq. Nature Biotech. 31, 46-53 (2012).

All references referred to or cited herein are incorporated herein in their entirety.

We claim:

1. A method of rejuvenating aged skeletal muscle stem cells of the niche of a subject that are defective in β1-integrin activity and unable to support FGF signaling, comprising administering to the niche of said subject an anti-CD29 antibody, such as a human or humanized antibody, chimeric or functional fragment or variant thereof, and/or RGD-containing peptides, such as fibronectin, fragment and/or variant thereof, that activate β1-integrin and FGF signaling restored, such that said aged skeletal muscle stem cells are rejuvenated.

2. The method of claim 1 wherein the skeletal muscle of the subject has been injured and said rejuvenated aged skeletal muscle stem cells support muscle regeneration after injury.

3. Use of an anti-CD29 antibody, such as a human or humanized antibody, chimeric or functional fragment or variant thereof, and/or an RGD-containing peptide or fragment or variant thereof, that activate β1-integrin to restore FGF signaling of aged skeletal muscle stem cells of the niche of a subject in need thereof.

4. A composition comprising an anti-CD29 antibody, such as a human or humanized antibody, chimeric or functional fragment or variant thereof, and/or RGD-containing peptides, such as fibronectin, fragment and/or variant thereof, that activates β1-integrin when contacted with aged skeletal muscle stem cells of the niche of a subject and restores FGF signaling such that said aged skeletal muscle stem cells are rejuvenated.

5. The composition of claim 4 wherein said anti-CD29 antibody, such as a human or humanized antibody, chimeric or functional fragment or variant thereof, comprises at least one of the VH CDRs and VL CDRs of monoclonal antibody TS2/16.

6. The composition of claim 4 wherein said anti-CD29 antibody, such as a human or humanized antibody, chimeric or functional fragment or variant thereof, comprises at least one of the VH CDRs and VL CDRs of monoclonal antibody 8A2.

7. The composition of claim 4 wherein said anti-CD29 antibody, such as a human or humanized antibody, chimeric or functional fragment or variant thereof, comprises at least one of the VH CDRs and VL CDRs of monoclonal antibody AIA5.
